SEQUENCE LISTING (132 sequences)

SEQ ID NO: 1 - DNA, 27 bp, Artificial Sequence
  Primer sequence
gaaataagttatagagagataaagatt

SEQ ID NO: 2 - DNA, 25 bp, Artificial Sequence
  Primer sequence
aaaaaaataaaataaataaaaaaaa

SEQ ID NO: 3 - DNA, 25 bp, Artificial Sequence
  Primer sequence
aaatttatagggaaagttttgatgg

SEQ ID NO: 4 - DNA, 28 bp, Artificial Sequence
  Primer sequence
tcaaataaaattcttacaaaaaaacaac

SEQ ID NO: 5 - DNA, 24 bp, Artificial Sequence
  Primer sequence
tttrrtttttttgtgttttggttt

SEQ ID NO: 6 - DNA, 24 bp, Artificial Sequence
  Primer sequence
tttggaagcaayaagtatyggctt

SEQ ID NO: 7 - DNA, 25 bp, Artificial Sequence
  Primer sequence
tggatatattgatttagagtatgtt

SEQ ID NO: 8 - DNA, 25 bp, Artificial Sequence
  Primer sequence
ataccatttaatacaaattaacctc

SEQ ID NO: 9 - DNA, 21 bp, Artificial Sequence
  Primer sequence
tttgtttgaatttrrtttttt

SEQ ID NO: 10 - DNA, 24 bp, Artificial Sequence
  Primer sequence
tgcaagcaanaanaagtaanntnt

SEQ ID NO: 11 - DNA, 25 bp, Artificial Sequence
  Primer sequence
taaattttttaggtgaatttttagg

SEQ ID NO: 12 - DNA, 25 bp, Artificial Sequence
  Primer sequence
tatcaaaaaatattactccaaatcc

SEQ ID NO: 13 - DNA, 25 bp, Artificial Sequence
  Primer sequence
ttttttttattattttgtatgtttt

SEQ ID NO: 14 - DNA, 25 bp, Artificial Sequence
  Primer sequence
aaattcacctaaaaaatttaatacc

SEQ ID NO: 15 - DNA, 20 bp, Artificial Sequence
  Primer sequence
tttttaaaaaattgattatt

SEQ ID NO: 16 - DNA, 21 bp, Artificial Sequence
  Primer sequence
aattgcatgctttttttttaa

SEQ ID NO: 17 - DNA, 23 bp, Artificial Sequence
  Primer sequence
ttgtatgttttgcrttttttata

SEQ ID NO: 18 - DNA, 21 bp, Artificial Sequence
  Primer sequence
atgcatggctyattagaaata

SEQ ID NO: 19 - DNA, 30 bp, Artificial Sequence
  Primer sequence
ttttatttttaaaagtaattataatgatta

SEQ ID NO: 20 - DNA, 25 bp, Artificial Sequence
  Primer sequence
aaatcttaacaaaatccaaaaccaa

SEQ ID NO: 21 - DNA, 26 bp, Artificial Sequence
  Primer sequence
tgagagattattttgtttttttttta

SEQ ID NO: 22 - DNA, 23 bp, Artificial Sequence
  Primer sequence
aaatttttaattttttaattgag

SEQ ID NO: 23 - DNA, 30 bp, Artificial Sequence
  Primer sequence
aaaattataaatagaagtgttatttaagtg

SEQ ID NO: 24 - DNA, 26 bp, Artificial Sequence
  Primer sequence
aaattcaattattcaactacctaaac

SEQ ID NO: 25 - DNA, 25 bp, Artificial Sequence
  Primer sequence
aagttttttttaaaataatggagat

SEQ ID NO: 26 - DNA, 25 bp, Artificial Sequence
  Primer sequence
aaaaataaaaaacaaaataaaaacc

SEQ ID NO: 27 - DNA, 26 bp, Artificial Sequence
  Primer sequence
tttgtaatttgtgtagttggtaataa

SEQ ID NO: 28 - DNA, 25 bp, Artificial Sequence
  Primer sequence
ataactctccataaaaaaactttcc

SEQ ID NO: 29 - DNA, 25 bp, Artificial Sequence
  Primer sequence
ttaatttttggaagttaagaaataa

SEQ ID NO: 30 - DNA, 25 bp, Artificial Sequence
  Primer sequence
caactaaccaataaactataaaaac

SEQ ID NO: 31 - DNA, 25 bp, Artificial Sequence
  Primer sequence
ttattttagagagttttgaaggtgt

SEQ ID NO: 32 - DNA, 30 bp, Artificial Sequence
  Primer sequence
attacccatcttacttattatcttcttcta

SEQ ID NO: 33 - DNA, 25 bp, Artificial Sequence
  Primer sequence
ttaattttttatggataggatttaa

SEQ ID NO: 34 - DNA, 30 bp, Artificial Sequence
  Primer sequence
aacactattaaacctaaaaactttaactaa

SEQ ID NO: 35 - DNA, 26 bp, Artificial Sequence
  Primer sequence
tttgatttgtataaaaattattttaa

SEQ ID NO: 36 - DNA, 25 bp, Artificial Sequence
  Primer sequence
aaaataaaaccaataactctatttc

SEQ ID NO: 37 - DNA, 24 bp, Artificial Sequence
  Primer sequence
ttnananaanaanaanaantaaga

SEQ ID NO: 38 - DNA, 21 bp, Artificial Sequence
  Primer sequence
tatgtgarctaggrcatggta

SEQ ID NO: 39 - DNA, 26 bp, Artificial Sequence
  Primer sequence
tgaaggtataggattaagtagaagtt

SEQ ID NO: 40 - DNA, 25 bp, Artificial Sequence
  Primer sequence
attatcccaaaatacataaaaaaaa

SEQ ID NO: 41 - DNA, 21 bp, Artificial Sequence
  Primer sequence
ttaytttgtaatgttgaayaa

SEQ ID NO: 42 - DNA, 24 bp, Artificial Sequence
  Primer sequence
ttttgtattaraaraarggtgttt

SEQ ID NO: 43 - DNA, 25 bp, Artificial Sequence
  Primer sequence
caaacttcttaggtgaatcctcagg

SEQ ID NO: 44 - DNA, 25 bp, Artificial Sequence
  Primer sequence
atagttttccacaacgaggtttagg

SEQ ID NO: 45 - DNA, 26 bp, Artificial Sequence
  Primer sequence
cttcttctactatcttgcatgtcttg

SEQ ID NO: 46 - DNA, 24 bp, Artificial Sequence
  Primer sequence
aaacttcttaggtgaatcctcagg

SEQ ID NO: 47 - DNA, 20 bp, Artificial Sequence
  Primer sequence
tcttcaaaaaattgaccatt

SEQ ID NO: 48 - DNA, 21 bp, Artificial Sequence
  Primer sequence
aactgcatgctctctttctaa

SEQ ID NO: 49 - DNA, 25 bp, Artificial Sequence
  Primer sequence
ccaatctttggaagccaagaaacaa

SEQ ID NO: 50 - DNA, 25 bp, Artificial Sequence
  Primer sequence
gttgaccggttactcgacattctcg

SEQ ID NO: 51 - DNA, 25 bp, Artificial Sequence
  Primer sequence
ccatcttagagagtcttgaaggtgc

SEQ ID NO: 52 - DNA, 30 bp, Artificial Sequence
  Primer sequence
caacgggtagaacgaataatagaagaagac

SEQ ID NO: 53 - DNA, 20 bp, Artificial Sequence
  Primer sequence
aaagcaatcgatgccacaac

SEQ ID NO: 54 - DNA, 19 bp, Artificial Sequence
  Primer sequence
agtccgcaaactgcatcac

SEQ ID NO: 55 - DNA, 22 bp, Artificial Sequence
  Primer sequence
catcactaccatggaacaaacc

SEQ ID NO: 56 - DNA, 22 bp, Artificial Sequence
  Pimer sequence
ctgttggatggaacttgctatg

SEQ ID NO: 57 - DNA, 23 bp, Artificial Sequence
  Primer sequence
gctcattattacgggaaatgtaa

SEQ ID NO: 58 - DNA, 19 bp, Artificial Sequence
  Primer sequence
cccggccttcttcttgaaa

SEQ ID NO: 59 - DNA, 20 bp, Artificial Sequence
  Primer sequence
gaaacgaggtttacggcaga

SEQ ID NO: 60 - DNA, 23 bp, Artificial Sequence
  Primer sequence
gggaccaacagaaacttatcaaa

SEQ ID NO: 61 - DNA, 22 bp, Artificial Sequence
  Primer sequence
aaaagatattggtggttcgatg

SEQ ID NO: 62 - DNA, 27 bp, Artificial Sequence
  Primer sequence
aagcaaataatcataacatctaaaagg

SEQ ID NO: 63 - DNA, 21 bp, Artificial Sequence
  Primer sequence
ctccaagcatttggaagagtg

SEQ ID NO: 64 - DNA, 21 bp, Artificial Sequence
  Primer sequence
ccgcttgttcaaaacctacaa

SEQ ID NO: 65 - DNA, 25 bp, Artificial Sequence
  Primer sequence
ccatcttagagagtcttgaaggtgc

SEQ ID NO: 66 - DNA, 30 bp, Artificial Sequence; identical to SEQ ID NO: 52
  Primer sequence

SEQ ID NO: 67 - DNA, 20 bp, Artificial Sequence
  Primer sequence
gtttctggtagtgcccgact

SEQ ID NO: 68 - DNA, 27 bp, Artificial Sequence
  Primer sequence
catagatatgcatgatacttttgatgt

SEQ ID NO: 69 - DNA, 20 bp, Artificial Sequence
  Primer sequence
tccaatctttggaagccaag

SEQ ID NO: 70 - DNA, 20 bp, Artificial Sequence
  Primer sequence
ggctaagaggcggttttctt

SEQ ID NO: 71 - DNA, 20 bp, Artificial Sequence
  Primer sequence
aaccaccaccagattcacca

SEQ ID NO: 72 - DNA, 20 bp, Artificial Sequence
  Primer sequence
gagtggtagttgcggtggaa

SEQ ID NO: 73 - DNA, 27 bp, Artificial Sequence
  Primer sequence
tcatcctaattaattttcactgacttg

SEQ ID NO: 74 - DNA, 21 bp, Artificial Sequence
  Primer sequence
ttcttcccccaccatatatcc

SEQ ID NO: 75 - DNA, 20 bp, Artificial Sequence
  Primer sequence
caactggccaatgagctgta

SEQ ID NO: 76 - DNA, 20 bp, Artificial Sequence
  Primer sequence
aagtcctcgaacacctcagc

SEQ ID NO: 77 - DNA, 22 bp, Artificial Sequence
  Primer sequence
aatattaacaccgtcgacgaaa

SEQ ID NO: 78 - DNA, 21 bp, Artificial Sequence
  Primer sequence
gctgaatttgttgagccagtt

SEQ ID NO: 79 - DNA, 20 bp, Artificial Sequence
  Primer sequence
gaagagcccccaaaatcttc

SEQ ID NO: 80 - DNA, 25 bp, Artificial Sequence
  Primer sequence
tccctacttgatctctgatcttgtt

SEQ ID NO: 81 - DNA, 21 bp, Artificial Sequence
  Primer sequence
ttttcgttgggagtttagtgc

SEQ ID NO: 82 - DNA, 20 bp, Artificial Sequence
  Primer sequence
tgttgagccagttcttctgc

SEQ ID NO: 83 - DNA, 21 bp, Artificial Sequence
  Primer sequence
cgctatacaacgaatccatga

SEQ ID NO: 84 - DNA, 20 bp, Artificial Sequence
  Primer sequence
tcacgggatgggtaaagaaa

SEQ ID NO: 85 - DNA, 20 bp, Artificial Sequence
  Primer sequence
ctaatgaacggacggtttgc

SEQ ID NO: 86 - DNA, 21 bp, Artificial Sequence
  Primer sequence
tgggctaaaataattgggaca

SEQ ID NO: 87 - DNA, 22 bp, Artificial Sequence
  Primer sequence
tgcgttaggactatccatttca

SEQ ID NO: 88 - DNA, 20 bp, Artificial Sequence
  Primer sequence
atgcaacatcgaatcatcca

SEQ ID NO: 89 - DNA, 23 bp, Artificial Sequence
  Primer sequence
tccatactttcacccaaaaagaa

SEQ ID NO: 90 - DNA, 23 bp, Artificial Sequence
  Primer sequence
tcttgcaacacaaaatgttaagg

SEQ ID NO: 91 - DNA, 27 bp, Artificial Sequence
  Primer sequence
tttaaactccatatctttgcagaaaac

SEQ ID NO: 92 - DNA, 25 bp, Artificial Sequence
  Primer sequence
ttcgtataaaccttaacgagagagc

SEQ ID NO: 93 - DNA, 21 bp, Artificial Sequence
  Primer sequence
cccatcccgtgatttattttt

SEQ ID NO: 94 - DNA, 20 bp, Artificial Sequence
  Primer sequence
ttacccaaccatttttgcac

SEQ ID NO: 95 - DNA, 20 bp, Artificial Sequence
  Primer sequence
tttctccggttacgttccac

SEQ ID NO: 96 - DNA, 20 bp, Artificial Sequence
  Primer sequence
tccgacagctgcatctacac

SEQ ID NO: 97 - DNA, 20 bp, Artificial Sequence
  Primer sequence
caagtgaaacgaggttacgg

SEQ ID NO: 98 - DNA, 20 bp, Artificial Sequence
  Primer sequence
gtacaaagttctcgccgtgt

SEQ ID NO: 99 - DNA, 23 bp, Artificial Sequence
  Primer sequence
tttacggcagaagacatagcaaa

SEQ ID NO: 100 - DNA, 25 bp, Artificial Sequence
  Primer sequence
tcatcacattgtcaatagattggag

SEQ ID NO: 101 - DNA, 20 bp, Artificial Sequence
  Primer sequence
tggaacgtaaccggagaaag

SEQ ID NO: 102 - DNA, 20 bp, Artificial Sequence
  Primer sequence
acgttgtttcttggcttcca

SEQ ID NO: 103 - DNA, 20 bp, Artificial Sequence
  Primer sequence
cggagaaagcaaatgaacga

SEQ ID NO: 104 - DNA, 21 bp, Artificial Sequence
  Primer sequence
ccacaactcctcctatgatcg

SEQ ID NO: 105 - DNA, 20 bp, Artificial Sequence
  Primer sequence
tccgagatcacatgttccaa

SEQ ID NO: 106 - DNA, 25 bp, Artificial Sequence
  Primer sequence
tcatcacattgtcaatagattggag

SEQ ID NO: 107 - DNA, 20 bp, Artificial Sequence
  Primer sequence
attccgatagtcgaccaagc

SEQ ID NO: 108 - DNA, 20 bp, Artificial Sequence
  Primer sequence
tgcgaaataccgaacatcaa

SEQ ID NO: 109 - DNA, 20 bp, Artificial Sequence
  Primer sequence
cccgacatatctgtgggaac

SEQ ID NO: 110 - DNA, 20 bp, Artificial Sequence
  Primer sequence
tggggtcccaatccttttac

SEQ ID NO: 111 - DNA, 22 bp, Artificial Sequence
  Primer sequence
ggaattctcaccccccagtaac

SEQ ID NO: 112 - DNA, 19 bp, Artificial Sequence
  Primer sequence
gggtcccaatccttttaca

SEQ ID NO: 113 - DNA, 19 bp, Artificial Sequence
  Primer sequence
gggtccagagcttgagact

SEQ ID NO: 114 - DNA, 20 bp, Artificial Sequence
  Primer sequence
cccttctccaaggattgaca

SEQ ID NO: 115 - DNA, 20 bp, Artificial Sequence
  Primer sequence
gtctttgagccgttgctttc

SEQ ID NO: 116 - DNA, 20 bp, Artificial Sequence
  Primer sequence
gtgaagctgcttttcccaag

SEQ ID NO: 117 - DNA, 37 bp, Artificial Sequence
  Primer sequence
aagtggcacttcatcgtctcccaatcaaaatgaagct

SEQ ID NO: 118 - RNA, 31 nt, Artificial Sequence
  Antisense probe
cuucugccguaaaccucguuucacuugaaaa

SEQ ID NO: 119 - RNA, 32 nt, Artificial Sequence
  Antisense probe
uuaagugcucguucauuugcuuucuccgaaaa

SEQ ID NO: 120 - DNA, 17 bp, Artificial Sequence
  Primer sequence
ctcgtagactgcgtacc

SEQ ID NO: 121 - DNA, 16 bp, Artificial Sequence
  Primer sequence
gacgatgagtctcgat

SEQ ID NO: 122 - DNA, 15 bp, Artificial Sequence
  Primer sequence
aattggtacgcagtc

SEQ ID NO: 123 - DNA, 14 bp, Artificial Sequence
  Primer sequence
cgatcgagactcat

SEQ ID NO: 124 - DNA, 18 bp, Artificial Sequence
  Primer sequence
agactgcgtaccaattca

SEQ ID NO: 125 - DNA, 17 bp, Artificial Sequence
  Primer sequence
gatgagtctcgatcgga

SEQ ID NO: 126 - DNA, 18 bp, Artificial Sequence
  Primer sequence
agactgcgtaccattcac

SEQ ID NO: 127 - DNA, 19 bp, Artificial Sequence
  Primer sequence
gatgagtctcgatcggact

SEQ ID NO: 128 - DNA, 18 bp, Artificial Sequence
  Primer sequence
agactgcgtaccattcaa

SEQ ID NO: 129 - DNA, 19 bp, Artificial Sequence
  Primer sequence
gatgagtctcgatcggaat

SEQ ID NO: 130 - DNA, 18 bp, Artificial Sequence
  Primer sequence
agactgcgtaccattcag

SEQ ID NO: 131 - DNA, 19 bp, Artificial Sequence
  Primer sequence
gatgagtctcgatcggatc

SEQ ID NO: 132 - DNA, 18 bp, Artificial Sequence
  Primer sequence
agactgcgtaccattcat

(19) United States
(12) Patent Application Publication
(10) Pub. No.: US 2013/0305415 A1
(43) Pub. Date: Nov. 14, 2013

(54) Method for Producing a Stress Tolerant Plant or Precursor Thereof

(71) Applicants: Michael James Wilkinson, Cwmystwyth (GB); Carlos Marcelino Rodriguez Lopez, Aberystwyth (GB); Penelope Tricker, Reading (GB); Paul Hadley, Reading (GB)

(72) Inventors: Michael James Wilkinson, Cwmystwyth (GB); Carlos Marcelino Rodriguez Lopez, Aberystwyth (GB); Penelope Tricker, Reading (GB); Paul Hadley, Reading (GB)

(73) Assignee: Aberystwyth University, Aberystwyth (GB)

(21) Appl. No.: 13/704,586

(22) PCT Filed: Jun. 14, 2011
(86) PCT No.: PCT/GB11/00904

§ 371 Date: Apr. 19, 2013

(30) Foreign Application Priority Data
Jun. 14, 2010 (GB) 1009945.5

(51) Int. Cl. A01H 3/00

(52) U.S. Cl. CPC A01H 3/00
USPC 800/298; 435/6.11

(57) ABSTRACT

Provided are methods for the production of a stress tolerant plant or precursor thereof. The methods comprise (i) subjecting one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation; and (ii) generating offspring from said one or more parental plants. The offspring show enhanced tolerance relative to the one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation. Also provided are plants, or precursor thereof, produced by the methods and assays for identifying a plant, or precursor thereof, produced by the methods.

### BACKGROUND OF THE INVENTION

The present application relates to methods for increasing tolerance of plants and/or their offspring to one or more stresses, in particular relating to the generation of stress tolerant plants and seeds/propagules thereof.

The ability to provide plants that are tolerant to usually unfavourable environmental conditions is highly desirable. For example, seeds that germinate into crop plants that show enhanced tolerance to drought or other water stresses than their parent(s) could be particularly useful. Moreover, it would be desirable to produce tolerant seeds/propagules for use in climates where yields are currently restricted by limited water availability.

### SUMMARY OF THE INVENTION

According to one aspect of the present invention, there is provided a method for the production of a stress tolerant plant or precursor thereof, the method comprising:

(i) subjecting one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation; and

(ii) generating offspring from said one or more parental plants,

wherein said offspring show enhanced tolerance relative to the one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation.

Preferably, the offspring are adult plants or a precursor thereof such as seeds or vegetative propagules.

Remarkably, it has been found that by subjecting a parent plant to one or more stress conditions, the seed or vegetative offspring produced from that parent plant can exhibit increased tolerance to the same one or more stress conditions. It has also, remarkably, been found that the offspring may be tolerant to one or more stress conditions which differ from those experienced by the one or more parental plants. For example Arabidopsis plants exposed to slightly reduced relative humidity stress nevertheless exhibited increased tolerance to periodic drought stress (See example 2 below). Accordingly, in one embodiment, it is preferred that the offspring are tolerant to one or more stress conditions not experienced by the one or more parental plants.

It will be appreciated that the term “unfavourable” is relative to the plant in question and is a relative term. For example, for plants which usually thrive in medium or high levels of humidity, a low relative humidity may be seen as “unfavourable” and therefore as a stress condition. For example, in the context of a food crop, any condition that leads to reduction in yields, harvestable yields or in sustainable harvests may be viewed as “unfavourable”.

Preferably, the one or more stress conditions are selected from low relative humidity, periodic drought and infection with Botrytis (e.g. Botrytis cynerea).

Preferably, the one or more parental plants are subjected to the one or more stress conditions under semi-controlled or more preferably, controlled conditions.

Preferably, the one or more parental plants are selected from a higher plant, a flowering plant and a dicotyledonous plant.

Preferably, the one or more parental plants are crop plants.

Preferably, the one or more parental plants belong to the Eudicotyledons. Preferably, the one or more parental plants are a member of the Brassicacea or the Malvaceae. Preferably, the one or more parental plants are selected from Arabidopsis plants and a Theobroma plants, for example selected from Arabidopsis thaliana and Theobroma cacao.

Preferably, the one or more parental plants are flowering plants (Magnoliophyta), and the one or more stress conditions are likely to impact on harvestable yield; for example including stresses associated with water availability (low relative humidity or periodic drought), to toxic chemicals (such as salt), exogenous chemicals or to exposure to pathogens (such as Botrytis) or pests.

Preferably, the methods of the present invention are for producing plants capable of generating higher yields under one or more stress conditions experienced and/or not experienced by the one or more parental plants. For example, it is preferred that the plants produced by the methods of the present invention show increased production of biomass, flower number, seed number, seed weight at any chosen time of harvest.

As described above, the methods of the present invention can be used to produce a precursor of a stress tolerant plant such as a seed or a vegetative propagule. For example, in one aspect of the present invention there is provided a method which comprises:

(i) subjecting one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation;

(ii) generating a precursor for offspring from said one or more parental plants, wherein said precursor is capable of developing into a plant which shows increased tolerance relative to the one or more parental plants to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation and/or is capable of generating higher yields relative to the one or more parental plants under said stress conditions.

Preferably, the precursor is a seed or a vegetative propagule such as a cutting. For example, the precursor may be an Arabidopsis seed which is capable of growing into a plant tolerant to low relative humidity and/or to periodic drought. In other examples, the precusor is a cutting or a somatic embryo of cocoa (Theobroma cacao L.) which is capable of growing into a plant tolerant to low relative humidity and/or to periodic drought. Further examples include an Arabidopsis seed which is capable of growing into a plant with enhanced resistance to Botrytis.

Preferably, the methods of the invention comprise crossing (i.e. cross-pollinating) two parental plants or self-pollinating a single parental plant. In other examples, a vegetative propagule is created from a parental plant that has been exposed to one or more stress conditions.

Preferably, the methods of the invention comprise generating seed offspring from a single parent genotype, for example by self-pollination or by cross-pollinating one of the treated parental plants with a second (untreated) parental plant.

It will be appreciated that subjecting a parental plant to one or more stress conditions includes subjecting all or a part of the plant to one or more stress conditions. For example, in the case of low relative humidity, all of the plant could be exposed. In the case of infection with Botrytis, a single leaf or portion thereof could be exposed.

According to another aspect of the present invention, there is provided a plant, or precursor thereof, produced by a method as described herein.

As such, the present invention provides a plant or precursor thereof which is tolerant to one or more stress conditions.

Another aspect of the present invention relates to an assay for identifying a plant, or precursor thereof, produced by the methods described herein, the assay comprising analysing a plant, or precursor thereof, suspected of being produced by the method for the presence or absence of one or more sites of genomic methylation, wherein the presence or absence of methylation at said one or more sites is indicative of a plant, or precursor thereof, produced by the method.

Preferably, the method is for producing a low relative humidity and/or periodic drought-tolerant plant (for example an Arabidopsis plant), or seed thereof, and the presence of a methylation state at or within about 10 kb, preferably about 5 kb, preferably about 2 kb of a SPEECHLESS or FAMA gene, or a functional homolog of either gene, is indicative of the acquired stress-tolerance in a plant or seed produced by the methods described herein.

Another aspect of the present invention provides an assay for identifying a plant, or precursor thereof, which is tolerant to one or more stress conditions selected from unfavourable conditions relating to relative humidity, water availability, periodic drought, nutrients, sunlight, wind, temperature, pH, exogenous chemicals, chemical toxins such as salt, herbivory, prophylactic chemicals, fertilizers, pathogen attack such as bacterial, fungal, or virus infection and pest infestation, wherein the assay comprises analysing a plant, or precursor thereof for the presence or absence of one or more sites of genomic DNA methylation, wherein the presence or absence of methylation at said one or more sites is indicative of a plant, or precursor thereof, which is tolerant to said one or more stress conditions. Preferably, the presence of genomic methylation in or within about 10 kb, preferably about 5 kb, preferably about 2 kb, of a SPEECHLESS or FAMA gene, or a functional homolog of either gene, is indicative of a plant, or precursor thereof, which is tolerant to low relative humidity and/or periodic drought.

It will be seen that, according to the present invention, there is provided a method for changing the stress response of the offspring of a plant by previously exposing (prior to conception/zygote formation) one or more of its parents to the same stress(es) or a different stress (hereafter also referred to as conditioning stress[es]).

As detailed herein, in some embodiments the change to stress response in the offspring relates to a different stress type to that experienced by the parents. That is, where exposure to the conditioning stress evokes a changed response to another stress in the offspring.

Preferably, the offspring are clonal propagules of a parental plant. Put another way, it is preferred that the change in stress response is induced in a clonal propagule of the parental plant exposed to the conditioning stress(es).

As will be appreciated, the seeds of crop plants in which either or both parents have been exposed to one or more conditioning stresses are produced for the purpose of improving the stress tolerance of the plants derived from said seeds.

As will be further appreciated, in accordance with the methods of the present invention, plants which have been exposed to one or more conditioning stresses can be used to produce vegetative propagules, for example cuttings, micropropagation, callus-mediated adentitious shooting or somatic embryogenesis, with changed, preferably improved, tolerance to one or more stress conditions.

Particularly preferred examples of the invention include the following.

Preferably, the seeds of plants in which either or both parents have been exposed to low relative humidity stresses are produced for the purpose of changing (preferably improving) the tolerance of the plants derived from said seeds to water stress (examples include but are not limited to low relative humidity stress and periodic drought).

Preferably, the seeds of plants in which either or both parents have been exposed to low relative humidity stresses are produced for the purpose of changing (preferably improving) the tolerance of the plants derived from said seeds to low relative humidity stress.

Preferably, the seeds of Eudicotyledonous plants in which either or both parents have been exposed to low relative humidity stresses are produced for the purpose of improving the tolerance of the plants derived from said seeds to water stress (examples include but are not limited to low relative humidity stress and periodic drought).

Preferably, the seeds of Eudicotyledonous plants in which either or both parents have been exposed to low relative humidity stresses are produced for the purpose of changing (preferably improving) the tolerance of the plants derived from said seeds to low relative humidity stress (examples include but are not limited to low relative humidity stress and periodic drought).

Preferably, the seeds of Brassicacea or Malvaceae plants in which either or both parents have been exposed to low relative humidity stresses are produced for the purpose of improving the tolerance of the plants derived from said seeds to water stress (examples include but are not limited to low relative humidity stress and periodic drought).

Preferably, the seeds of Brassicacea or Malvaceae plants in which either or both parents have been exposed to low relative humidity stresses are produced for the purpose of changing (preferably improving) the tolerance of the plants derived from said seeds to low relative humidity stress.

Preferably, the plants which have been exposed to low relative humidity stresses are used to produce vegetative propagules with changed (preferably improved) tolerance to water stress (examples include but are not limited to low relative humidity stress and periodic drought).

Preferably, the plants which have been exposed to low relative humidity stresses are used to produce vegetative propagules with changed (preferably improved) tolerance to low relative humidity stress.

Preferably, the seeds of plants in which either or both parents have been exposed to biotic stress (examples include but not limited to exposure to pathogenic fungi) are produced for the purpose of changing (preferably improving) the resistance of the plants derived from said seeds to the same biotic stresses.

Preferably, the seeds of eudicotyledonous plants in which either or both parents have been exposed to biotic stress (examples include but not limited to exposure to pathogenic fungi) are produced for the purpose of changing (preferably improving) the resistance of the plants derived from said seeds to the same biotic stresses.

Preferably, the seeds of plants of the Brassicacea or Malvaceae in which either or both parents have been exposed to biotic stress (examples include but not limited to exposure to pathogenic fungi) are produced for the purpose of changing (preferably improving) the resistance of the plants derived from said seeds to the same biotic stresses.

Preferably, the seeds of plants in which either or both parents have been exposed to Botrytis fungi are produced for the purpose of changing (preferably improving) the resistance of the plants derived from said seeds to infection by Botrytis fungi.

Preferably, the seeds of eudicotyledonous plants in which either or both parents have been exposed to Botrytis fungi are produced for the purpose of changing (preferably improving) the resistance of the plants derived from said seeds to infection by Botrytis fungi.

Preferably, the seeds of plants of the Brassicacea or Malvaceae in which either or both parents have been exposed to Botrytis fungi are produced for the purpose of changing (preferably improving) the resistance of the plants derived from said seeds to infection by Botrytis fungi.

Preferably, the changed stress responses of plants (preferably crop plants) whose parents have been exposed to conditioning stresses (as identified above) leads to changed (preferably enhanced) production of biomass, flower number, seed number, seed weight at any chosen time of harvest.

Preferably, plants with changed tolerance to water stress are produced according to the methods described herein are detected according to changed methylation status of the DNA (measured using standard techniques including but not limited to bisulfite treatment followed by Sanger or NextGen sequencing, High Resolution Melt Analysis or Methyl capture and pPCR) encoding for the SPEECHLESS and/or FAMA genes (or functional homologue thereof) and/or of the DNA sequence immediately flanking said gene, where flanking sequence is preferably <10 kb, more preferably <3 kb and most preferably <1.5 kb of start or stop codons.

Example embodiments of the present invention will now be described with reference to the accompanying figures in which:—

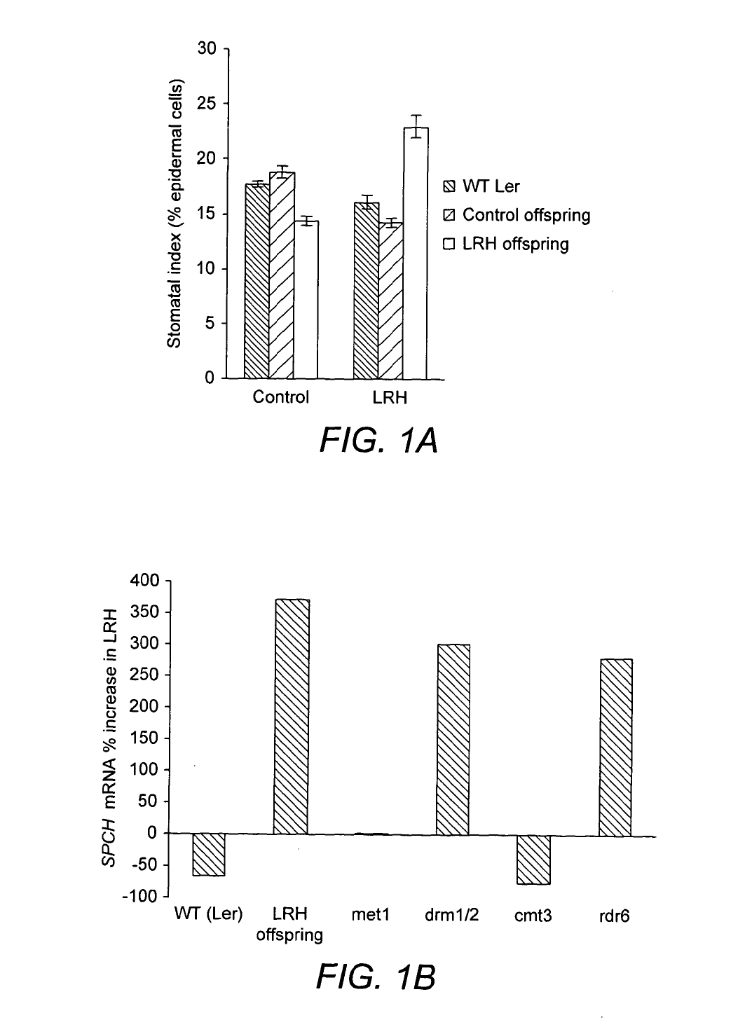

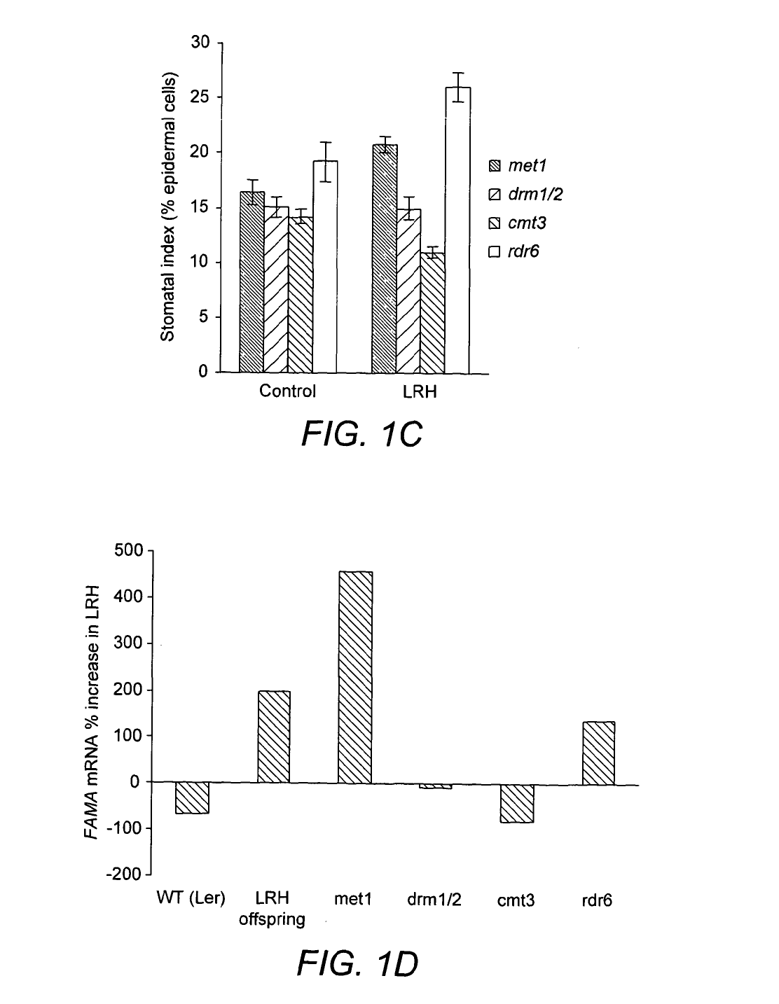

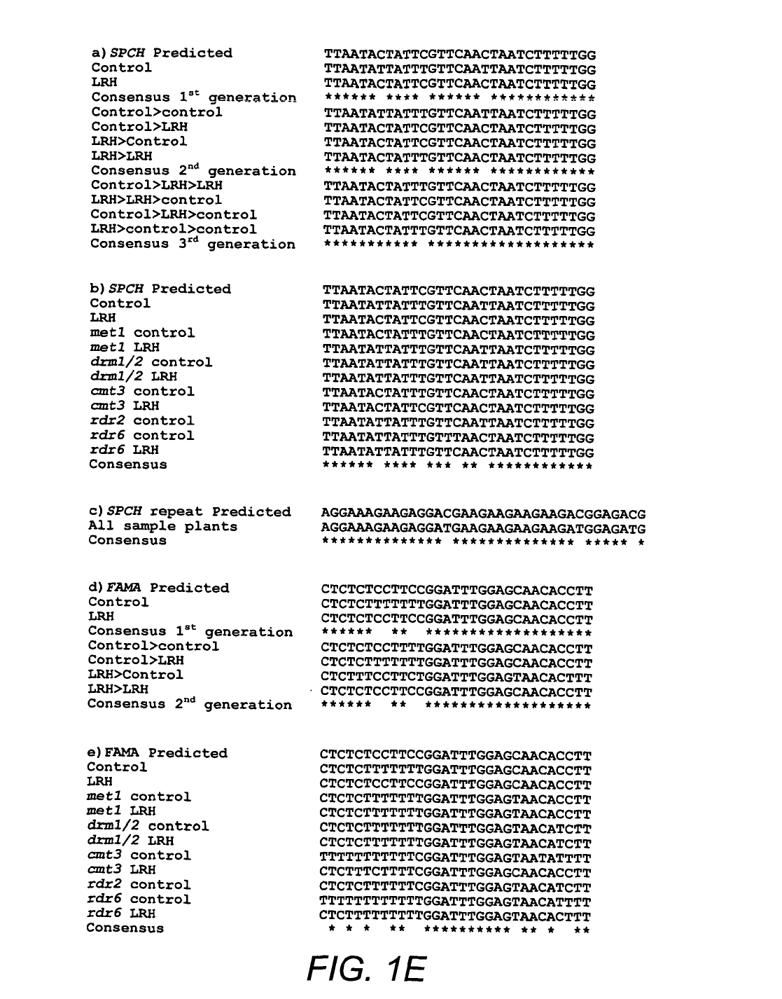

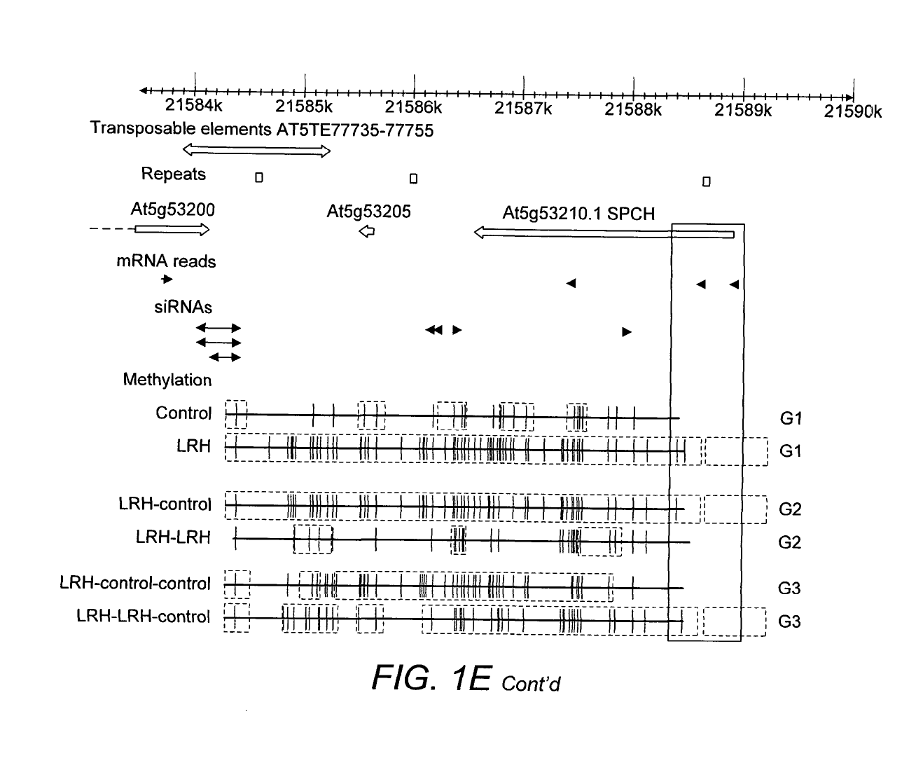

FIG. 1 shows that differential stomatal index (SI) with low relative humidity treatment*parent is positively correlated with expression of the SPCH and FAMA genes and inversely correlated with DNA methylation of SPCH. Wild type (WT) SI is reduced in low relative humidity (LRH): in the progeny of the LRH-treated parent grown in LRH, however, SI is increased (ANOVA: treatment P=0.001, parent P=<0.001, interaction P=<0.001). This effect is abolished in the methyltransferase mutants met1 and drm1/2. Data shown are means (±s.e.m.) for one experiment where n=48. Expression of the SPCH and FAMA stomata pathway genes is also reduced in the parent in LRH but not in the progeny; neither is expression of SPCH nor FAMA reduced significantly by LRH in met1, drm1/2 or the siRNA mutant rdr6. Data shown are mean percentage increases in [mRNAs] from three repeated assays for each target in LRH relative to their own control (0 line of the x axis). Sequences of samples following bisulfite conversion show de novo cytosine methylation with LRH at SPCH (a—consensus 1^{St} generation) and FAMA (d—consensus 1^{St} generation) which is not reproduced in the met1 or drm1/2 mutants (b and e). This methylation is heritable in SPCH but lost in the progeny of the LRH-grown parent grown in LRH (a—consensus 2^{nd} generation). The pattern of differential methylation with LRH stress at the SPCH locus (TAIR v. 9.0) is shown for three generations. G1 plants (first exposure) plants are de novo methylated in all contexts under LRH stress (additional 78 sites in 4.25 kb). Progeny of these plants (G2) inherit the majority of methylation (LRH-control) but are substantially demethylated when returned to LRH stress (LRH-LRH). There is a loss of inherited methylation in the next generation (G3, LRH-control-control) including at the upstream regulatory region and transcription start sites, but this region is remethylated heritably during a second exposure to stress (LRH-LRH-control). In FIG. 1E shaded areas (outlined by dashed lines) indicate methylated regions by methyl capture+qPCR in Chromosome 5 from 21584.3 k to 21589.3 k, and bars methylated base pairs by 454 sequencing following bisulfite conversion (green=CG, blue=CHG, pink=CHH contexts). Black horizontal lines show the extent of successful 454 sequencing and methods. The boxed region indicates the region assayed at base-pair resolution by sub-cloning and sequencing following bisulfite conversion and from which representative sequences are shown in a-c;

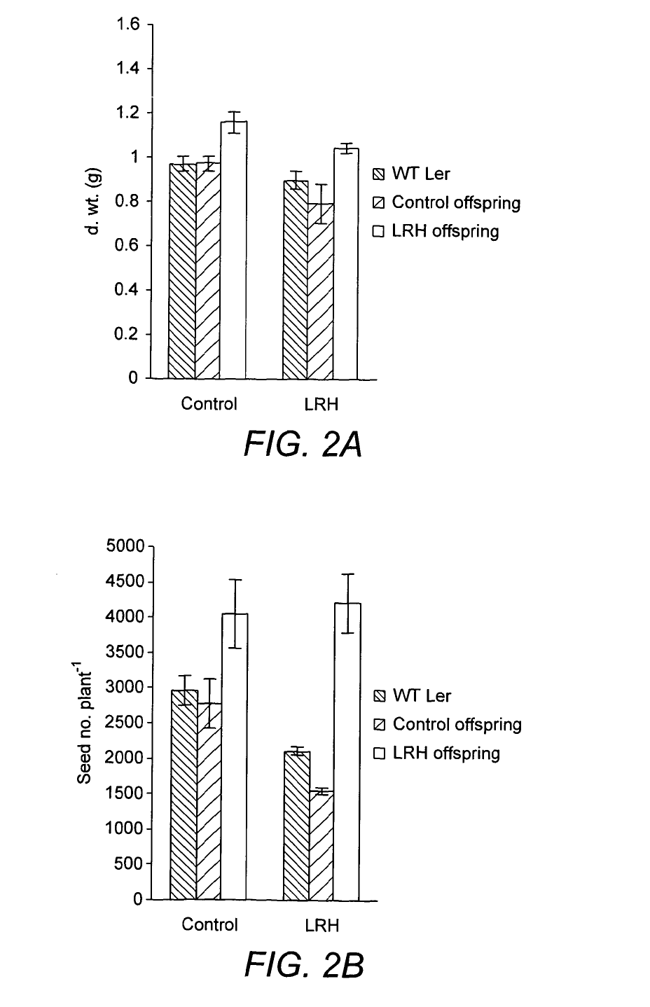

FIG. 2 shows that (A) the size (dry weight (g) at harvest) and (B) productivity (seed number produced) of progenies are also influenced by parental experience of stress. Offspring of parents exposed to LRH stress exhibited increased size and productivity in both the LRH treatment and in control conditions (ANOVA: treatment P=<0.001, parent P=<0.001, interaction P=0.39). This effect was characteristic of all the tested progeny plants in a line (n=48 in each repeated experiment) from all exposed parents (n=3 in each experiment) in four repeated experiments;

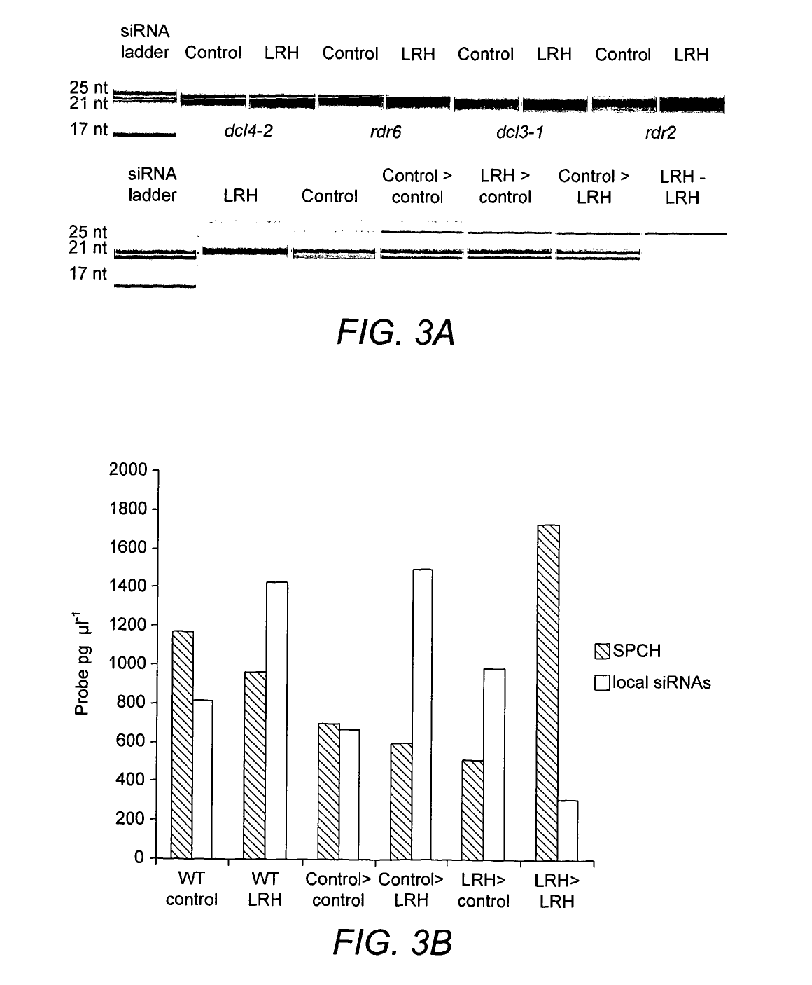

FIGS. 3A and 3B show siRNAs concentration in LRH treatment*parent experiments. There was an increase in the total concentration of 24 nt siRNAs on first exposure to LRH and a decrease when offspring of LRH-treated parents are grown under LRH stress. Induction of siRNAs at transposable elements upstream (in the genome) of SPCH was inversely correlated with gene expression and positively correlated with methylation at SPCH;

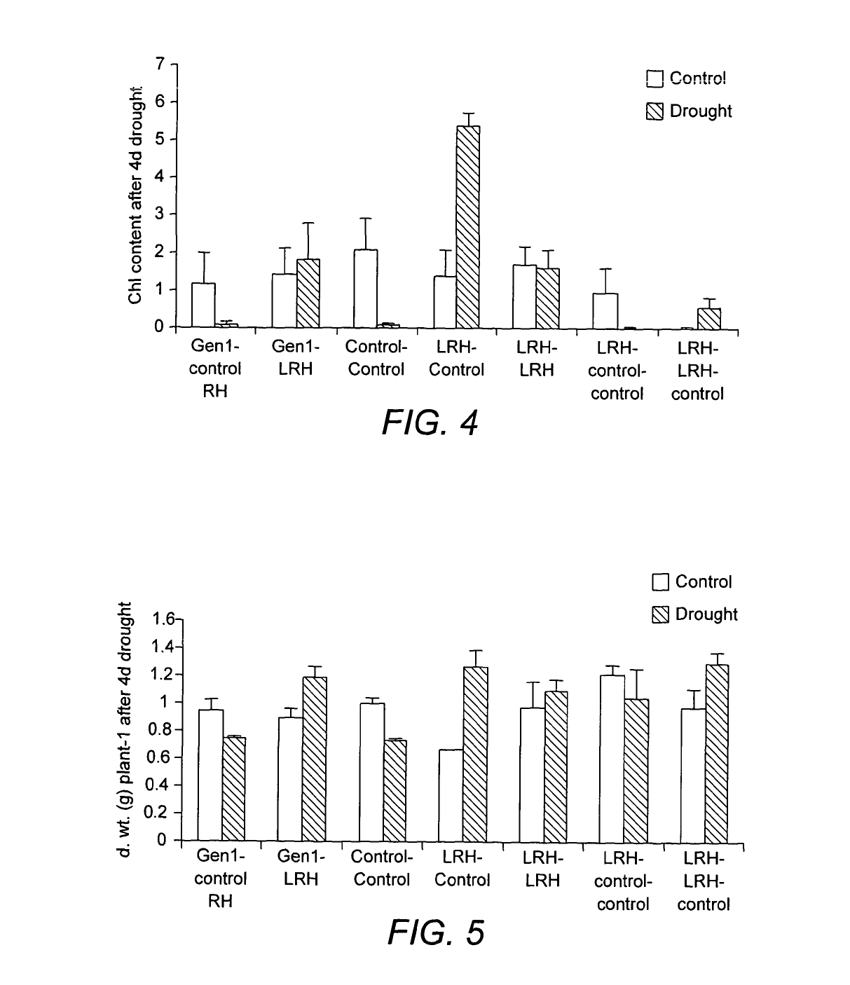

FIG. 4 shows the effect of preconditioning with Low Relative Humidity on chlorophyll content after 4 days drought. Offspring of parents exposed to LRH stress exhibited increased chlorophyll content in both the LRH treatment and when subjected to periodic drought;

FIG. 5 shows the effect of preconditioning with Low Relative Humidity on plant dry weight after 4 days drought. Offspring of parents exposed to LRH stress exhibited increased final dry weight in both the LRH treatment and when subjected to periodic drought;

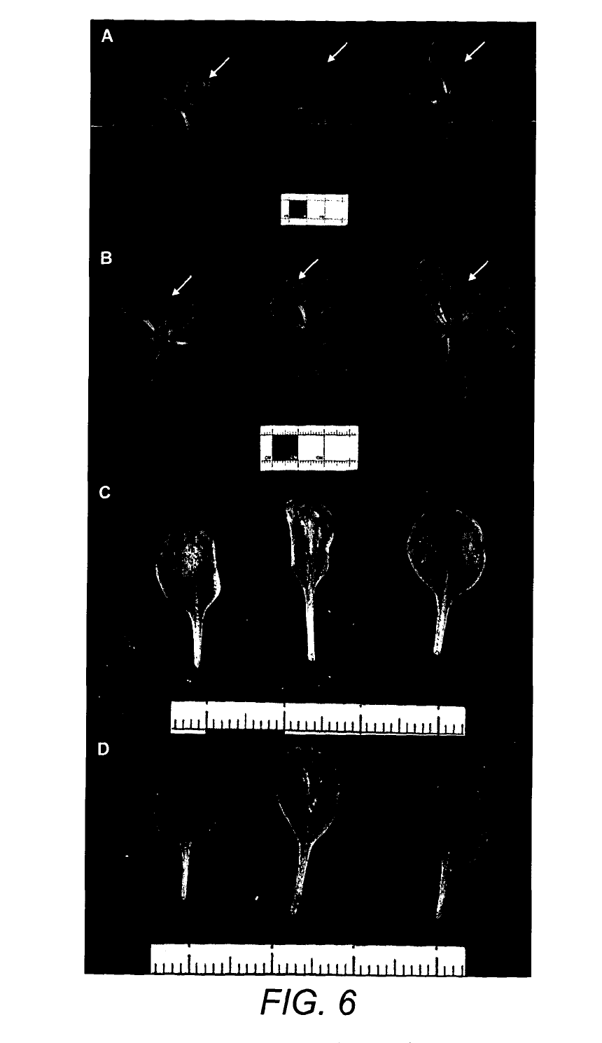

FIG. 6 shows resistance to Botrytis cynerea is increased by non-lethal innoculation on the previous generation. Generation 2 plants were treated with Botrytis cynerea. Pictures show lesions associated to fungal infection three days after inoculation in Langsberg erecta. Offspring of non-inoculated plants (A), offspring of inoculated plants (B) Arrows point inoculated leaves. Detail of the inoculated leaves from offspring of non-inoculated plants (C), offspring of inoculated plants (D);

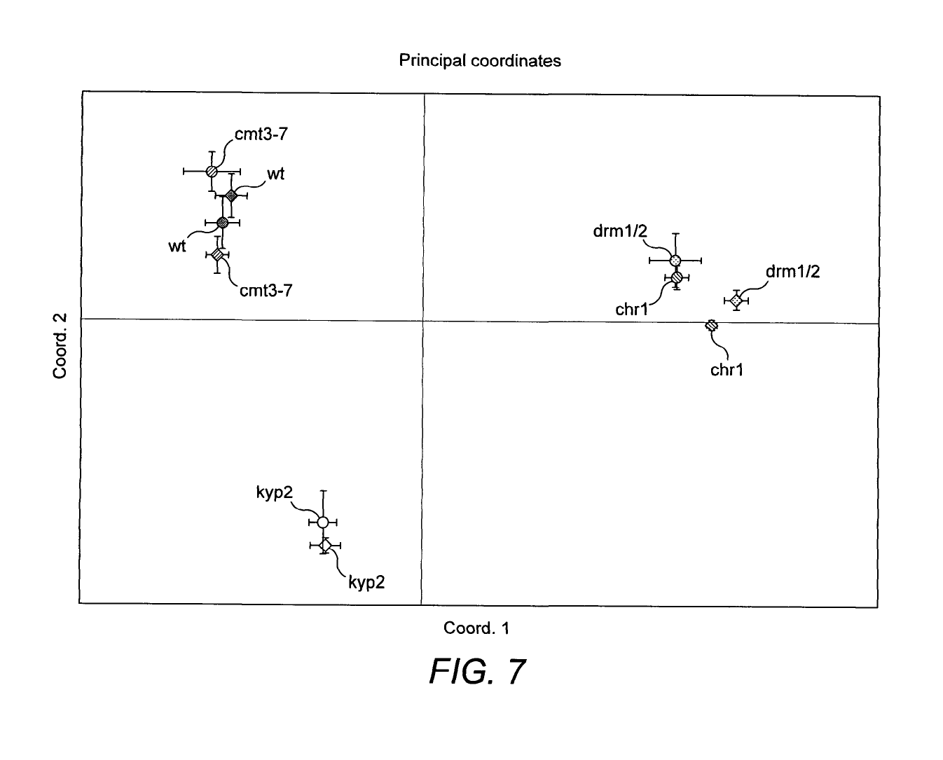

FIG. 7 shows analysis of global methylation changes induced by infection with Botrytis cynerea using restriction enzyme MspI. DNA from five different Arabidopsis thaliana genotypes (Wild type—Laer, and methylation mutants: drm1/2, chr1, cmt3-7 and kyp2) inoculated (rhomboids) and pathogen free (circles) (24 samples each) was restricted using the enzyme combination MspI/EcoRI (sensitive to methylation on the CpHpG motif). No significant differences were found between treatments. Error bars show calculated standard deviations; and

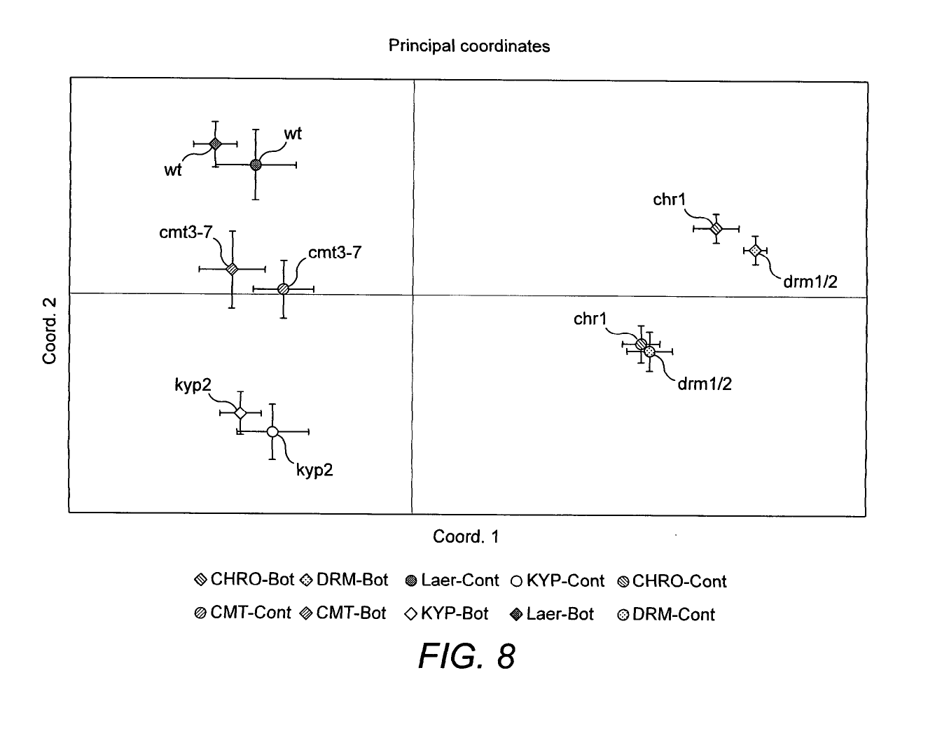

FIG. 8 shows analysis of global methylation changes induced by infection with Botrytis cynerea using restriction enzyme MspI. DNA from five different Arabidopsis thaliana genotypes (Wild type—Laer, and methylation mutants: drm1/2, chr1, cmt3-7 and kyp2) inoculated (rhomboids) and pathogen free (circles) (24 samples each) was restricted using the enzyme combination HpaII/EcoRI (sensitive to methylation on the CpHpG and the CpG motiffs). No differences were found between treatments for genotypes Wild type—Laer and kyp2. Genotype cmt3-7 showed some degree of separation (not significant) between samples infected with Botrytis and those non-infected. Genotypes drm1/2 and chr1 showed a significant on global DNA methylation induced by the infection with Botrytis. Error bars show calculated standard deviations.

### DETAILED DESCRIPTION OF THE INVENTION

The invention relates to methods for the production of plants and precursors thereof that are tolerant to one or more stress conditions. In particular, the invention relates to methods for producing seeds and/or vegetative propagules that have an enhanced ability to survive, grow and/or produce harvestable products when placed under one or more sub-optimal growing conditions (stresses) that in ‘parental’ plants and untreated lineages cause a significant drop in growth, survivorship, biomass, seed production and/or harvestable yield (for crops).

The methods used in the invention and detailed examples of the invention are set out below.

Within this specification, embodiments have been described in a way that enables a clear and concise specification to be written, but it is intended and will be appreciated that embodiments may be variously combined or separated without parting from the invention.

Within this specification, the terms “comprises” and “comprising” are interpreted to mean “includes, among other things”. These terms are not intended to be construed as “consists of only”.

Within this specification, the term “about” means plus or minus 20%, more preferably plus or minus 10%, even more preferably plus or minus 5%, most preferably plus or minus 2%.

Within this specification, the term “homolog” may mean a gene related to a second gene by descent from a common ancestral DNA sequence. The term may mean a gene similar in structure and evolutionary origin to a gene in another species.

Within this specification, the term “propagule” means any plant material which can be used for the purpose of plant propagation. In asexual reproduction, a propagule may be a woody, semi-hardwood, or softwood cutting, leaf section, or any number of other plant parts. In sexual reproduction, a propagule is a seed or spore. In micropropagation, a type of asexual reproduction, any part of the plant may be used, though it is usually a highly meristematic part such as root and stem ends or buds.

Within this specification, the term “vegetative propagule” means offspring which is the clonal (i.e. genetically identical) descendant of a single parental plant derived via plant materials other than a biological seed. This is in contrast to seed which is usually the result of sexual reproduction, i.e. the decendant of two or more parental plants.

Within this specification, the term “tolerant” means that the offspring/vegetative propagule(s) show an increased tolerance to one or more stresses than do the parental plant(s). It is preferred that this increase is statistically significant.

### Example 1

### Inherited Response to Low Humidity Stress in Arabidopsis Offspring

It has been found that Arabidopsis plants exposed to one form of water stress (low relative humidity LRH), respond in the short term by reducing the number of stomata (pores) in the leaves so that they do not lose excessive amounts of water and survive. The resultant plants are small but do nevertheless survive to set some seeds. Quite remarkably, however, seeds collected from these plants perform better when placed in identical conditions. The plants are also larger and produce far more seed. The plants used are so inbred that the offspring are to all intents and purposes genetically identical to the parent. Thus, it has remarkably been shown that stressing the parent plant pre-adapts the offspring to the same stress in the next generation. A similar phenomenon has been seen with other stresses (i.e. cold and heat stress (Whittle et al, 2009), UV-C light (Molinier et al, 2006), and pathogens such as bacteria (Molinier et al, 2006). The invention described herein is particularly applicable to commercial seed production in crops. Moreover, growing the parental clones/populations used to produce commercial seed lots under appropriate stressed conditions should pre-programme the epigenetic profiles of the seeds to increase the potential for adaptation to the same stresses when germinated. Importantly, such effects do not persist over many generations and rapidly fade. Thus, the present invention provides a means of improving plant production without changing the genetic code.

The density and operation (opening) of stomatal pores on the surface of leaves are both heavily influenced by environmental cues; together they control stomatal conductance of the leaf to water vapour (gₛ) over short (minute to hour) and long (seasonal to lifetime) timescales. Such plasticity allows the plant to balance the conflicting needs to capture atmospheric carbon dioxide (CO₂) for photosynthesis and to minimise water loss through transpiration and water use efficiency (wue) is inversely correlated with leaf stomatal density over a plant's lifetime. There have been recent advances in our understanding of the genetic regulation of stomatal development. A pathway governing stomatal guard cell development involves a “default” fate of protoderm epidermal cells to form stomata but expression of a series of patterning genes blocks entry into the stomatal lineage (and so guard cell formation) and consequently sets stomatal density. Positive regulators determine entry into the stomatal lineage and asymmetric divisions forming stomatal guard cells. Mechanisms allowing plants to maintain plasticity for water conservation and carbon fixation in response to the environmental cues they receive are less clear. The possibility that plastic responses to environmental stress experienced early in the life of the plant could provide adaptive conditioning in anticipation for similar stresses later in development or even in the seminal generation was investigated.

The response of the stomatal pathway to different levels of ambient humidity was analysed. Arabidopsis thaliana ecotypes Landsberg erecta and Columbia were grown under constant low relative humidity (LRH; 45%±5) or under experimental control (65%±5) humidity from seed to seed harvest. Stomatal frequency (index of stomata as a percentage of epidermal cells (SI)) was influenced by LRH stress (FIG. 1). In the Landsberg erecta ecotype, it was reduced in each repeated experiment with a large effect each time (Cohen's d-test >0.80) and wue increased. In isogenic progeny of the LRH-treated parent, however, SI was no longer reduced when progeny were exposed to the same LRH stress (LRH-LRH) (FIG. 1). Wue was no longer correlated with SI and increased. Likewise, fitness (measured as the parameters biomass and seed number) was reduced by LRH stress in the first exposed generation but not in LRH-LRH plants (FIG. 2). When stressed plants were returned to control RH during development (LRH→control) SI still decreased (25% decrease, P=0.028), and when stressed plants were crossed with control plants (LRHxcontrol) SI was increased under LRH stress (LRHxcontrol-LRH) compared with crosses from control plants (Controlxcontrol-LRH) (40% increase, P=<0.01). All sample plants were affected similarly.

DNA methylation of loci for genes in the stomata pathway was investigated to see whether it was imposed differentially with environment. Differences in DNA methylation were screened for under LRH compared with the control environment in 11 stomata patterning and formation genes (Table 1). Differential methylation associated with RH treatment was found in both regulatory and 5′ coding regions of the SPEECHLESS(SPCH) and FAMA genes (FIG. 1). SPCH and FAMA genes are paralogues encoding for basic-Helix Loop Helix (bHLH) proteins that are putative transcription factors. Expression of these genes, in a pathway with MUTE and their dimerization partners ICE1 and SCREAM2, regulates the entry of protoderm cells into the stomatal lineage and controls subsequent asymmetric and amplifying cell divisions culminating in the formation of stomatal guard cells. SPCH is required to initiate asymmetric cell divisions forming meristemoids and FAMA regulates the final division of the guard mother cell. SPCH and FAMA were significantly more methylated from leaves exposed to LRH. Methylation at the 5′ region of transcription factor genes is rare in the Arabidopsis genome and may be caused by or cause aberrant expression. Expression of both genes was suppressed under LRH conditions. Gene expression was suppressed (by 31-58%) (FIG. 1) when DNA was methylated under LRH and correlated with reduction in stomatal number on the leaf epidermis.

TABLE 1
Primer designs for bisulfite-specific PCR of genes in the stomata
pathway. ER, ERL1 and ERL2 were also tested in the Col-0 ecotype.

| Gene | GenBank | Primer | |
|---|---|---|---|
| name | accession no. | name | DNA sequence (5′-3′) |
| ER | At2g26330 | ERF | GAAATAAGTTATAGAGAGATAAAGATT (SEQ ID NO: 1) |
| ER | At2g26330 | ERR | AAAAAAATAAAATAAATAAAAAAAA (SEQ ID NO: 2) |
| ERL1 | At5g62230 | ERL1F | AAATTTATAGGGAAAGTTTTGATGG (SEQ ID NO: 3) |
| ERL1 | At5g62230 | ERL1R | TCAAATAAAATTCTTACAAAAAAACAAC (SEQ ID NO: 4) |
| ERL1 | At5g62230 | ERL1FD | TTTRRTTTTTTTGTGTTTTGGTTT (SEQ ID NO: 5) |
| ERL1 | At5g62230 | ERL1RD | TTTGGAAGCAAYAAGTATYGGCTT (SEQ ID NO: 6) |
| ERL2 | At5g07180 | ERL2F | TGGATATATTGATTTAGAGTATGTT (SEQ ID NO: 7) |
| ERL2 | At5g07180 | ERL2R | ATACCATTTAATACAAATTAACCTC (SEQ ID NO: 8) |
| ERL2 | At5g07180 | ERL2FD | TTTGTTTGAATTTRRTTTTTT (SEQ ID NO: 9) |
| ERL2 | At5g07180 | ERL2RD | TGCAAGCAAIAAIAAGTAAIITIT (SEQ ID NO: 10) |
| FAMA | At3g24140 | FAMAF | TAAATTTTTTAGGTGAATTTTTAGG (SEQ ID NO: 11) |
| FAMA | At3g24140 | FAMAR | TATCAAAAAATATTACTCCAAATCC (SEQ ID NO: 12) |
| FAMA | At3g24140 | FAMA2F | TTTTTTTTATTATTTTGTATGTTTTG (SEQ ID NO: 13) |
| FAMA | At3g24140 | FAMA2R | AAATTCACCTAAAAAATTTAATACC (SEQ ID NO: 14) |
| FAMA | At3g24140 | FAMAFp | TTTTTAAAAAATTGATTATT (SEQ ID NO: 15) |
| FAMA | At3g24140 | FAMARp | AATTGCATGCTTTTTTTTTAA (SEQ ID NO: 16) |
| FAMA | At3g24140 | FAMAFD | TTGTATGTTTTGCRTTTTTTATA (SEQ ID NO: 17) |
| FAMA | At3g24140 | FAMARD | ATGCATGGCTYATTAGAAATA (SEQ ID NO: 18) |
| FAMA | At3g24140 | FAMAFpr | TTTTATTTTTAAAAGTAATTATAATGATTA (SEQ ID NO: 19) |
| FAMA | At3g24140 | FAMARpr | AAATCTTAACAAAATCCAAAACCAA (SEQ ID NO: 20) |
| ICE1 | At3g26744.1 | ICE1F | TGAGAGATTATTTTGTTTTTTTTTTA (SEQ ID NO: 21) |
| ICE1 | At3g26744.1 | ICE1R | AAATTTTTAATTTTTTAATTGAG (SEQ ID NO: 22) |
| MUTE | At3g06120 | MUTEFpr | AAAATTATAAATAGAAGTGTTATTTAAGTG (SEQ ID NO: 23) |
| MUTE | At3g06120 | MUTERpr | AAATTCAATTATTCAACTACCTAAAC (SEQID NO: 24) |
| SCRM2 | At1g12860 | SCRMF | AAGTTTTTTTTAAAATAATGGAGAT (SEQ ID NO: 25) |
| SCRM2 | At1g12860 | SCRMR | AAAAATAAAAAACAAAATAAAAACC (SEQ ID NO: 26) |
| SDD1 | At1g04110 | SDD1F | TTTGTAATTTGTGTAGTTGGTAATAA (SEQ ID NO: 27) |
| SDD1 | At1g04110 | SDD1R | ATAACTCTCCATAAAAAAACTTTCC (SEQ ID NO: 28) |
| SPCH | At5g53210 | SPCHF | TTAATTTTTGGAAGTTAAGAAATAA (SEQ ID NO: 29) |
| SPCH | At5g53210 | SPCHR | CAACTAACCAATAAACTATAAAAAC (SEQ ID NO: 30) |
| SPCH | At5g53210 | SPCHFp | TTATTTTAGAGAGTTTTGAAGGTGT (SEQ ID NO: 31) |
| SPCH | At5g53210 | SPCHRp | ATTACCCATCTTACTTATTATCTTCTTCTA (SEQ ID NO: 32) |
| SPCH | At5g53210 | SPCHFpr | TTAATTTTTTATGGATAGGATTTAA (SEQ ID NO: 33) |
| SPCH | At5g53210 | SPCHRpr | AACACTATTAAACCTAAAAACTTTAACTAA (SEQ ID NO: 34) |
| TMM | At1g80080 | TMMF | TTTGATTTGTATAAAAATTATTTTAA (SEQ ID NO: 35) |
| TMM | At1g80080 | TMMR | AAAATAAAACCAATAACTCTATTTC (SEQ ID NO: 36) |
| TMM | At1g80080 | TMMFD | TTIAIAIAAIAAIAAIAAITAAGA (SEQ ID NO: 37) |
| TMM | At1g80080 | TMMRD | TATGTGARCTAGGRCATGGTA (SEQ ID NO: 38) |
| YDA | At1g63700 | YDAF | TGAAGGTATAGGATTAAGTAGAAGTT (SEQ ID NO: 39) |
| YDA | At1g63700 | YDAR | ATTATCCCAAAATACATAAAAAAAA (SEQ ID NO: 40) |
| YDA | At1g63700 | YDAFD | TTAYTTTGTAATGTTGAAYAA (SEQ ID NO: 41) |
| YDA | At1g63700 | YDARD | TTTTGTATTARAARAARGGTGTTT (SEQ ID NO: 42) |

The role of methylation in regulating stomatal frequency was further investigated using methyltransferase mutants. In the mutant for the maintenance cytosine methyltransferase MET1 (Decreased Methylation 2DNA) (met1)) SI was increased in LRH and differential methylation with treatment was reduced for both SPCH and FAMA. Expression of SPCH was no longer reduced by LRH treatment and FAMA expression increased (FIG. 1). In the double mutant for Domains Rearranged Methyltransferases 1 and 2 (drm1/drm2) SI was not reduced by LRH, nor was expression of FAMA, SPCH expression increased and no differential methylation was detected with treatment (FIG. 1). DOMAINS REARRANGED METHYLTRANSFERASE 2 (DRM2) is the only enzyme so far known to methylate DNA de novo in Arabidopsis. De novo methylation of SPCH and FAMA occurred in response to LRH stress. Single base-resolution sequences of fragments of the SPCH and FAMA gene loci showed differential methylation with treatment in both symmetric (CG) and asymmetric (CHH where H is any base) contexts (FIG. 1). Additional asymmetric methylation under LRH in the wild type (WT) was not imposed in the drm1/2 plants at either gene locus (FIG. 1). Non-CG methylation is maintained redundantly by DRM2 and the protein CHROMOMETHYLASE 3 (CMT3). We also examined the response of the cmt3 mutant and found that it responded to LRH in the same manner as the WT plants, with reduced SI, reduced expression and increased methylation in LRH (data not shown). LRH-induced asymmetric methylation of FAMA was abolished in the cmt3 plants. These differences between the two genes implied that de novo establishment of asymmetric methylation was required for the response in FAMA but that CG-dependent maintenance of induced methylation might be equally important for the differential environmental response of SPCH.

DRM2-mediated transcriptional gene silencing (TGS) by uni-directional methylation of gene promoter sequences in Arabidopsis is directed by 24 nt short-interfering RNAs (siRNAs): Post-transcriptional gene silencing (PTGS) by 21-22 nt secondary siRNAs has also been associated with bi-directional methylation of transcribed regions. A range of mutants for RNA-directed DNA methylation (RdDM) was grown in the control and LRH environments to investigate the role of siRNA direction in the observed DNA methylation and physiological responses. In TGS, RDR2 (ma dependent rna polymerase 2) is required for the synthesis of double-stranded short RNAs. Both maintenance and transitivity of PTGS require RDR6 and depend on transcription of the target gene. Dicer-like RNA III proteins process dsRNA or hairpin RNAs with DCL3 primarily acting on RDR2-produced RNAs and DCL4 on RDR6-produced RNAs; there is, however, some overlap and compensatory processing by the four Arabidopsis DCLs in single dcl mutants. No true rdr2 or dcl3 mutants germinated under LRH stress; both genes were expressed (data not shown), total small RNA content was increased compared with the WT and 24 nt siRNAs were present in seedlings although at much reduced levels (FIG. 3). In the control treatment, intriguingly, stomatal frequency of both the rdr2 and dcl3 mutants (compared with their background ecotype Columbia) was higher (data not shown), suggesting that siRNAs may be required to suppress the formation of stomata at some point in the pathway. Both SPCH and FAMA remained comparatively unmethylated at asymmetric bases in rdr2, as in drm1/2, (FIG. 1) and expression of FAMA was increased confirming previous observations. 21 nt siRNAs were present in dcl4 plants in both treatments but not at measurable levels in rdr6 (data not shown). LRH-induced methylation was reduced overall in rdr6 but not abolished in either symmetrical or asymmetrical contexts in SPCH or FAMA (FIG. 1). Expression of both SPCH and FAMA increased in rdr6 (FIG. 1, confirming previous results) and SI increased both in the WT control and under LRH. These data showed that both SPCH and FAMA were targets of RdDM under environmental stress and implied that both loci could be subject to TGS and PTGS.

Small RNA reads from high-throughput sequencing data of A. thaliana show seven small RNAs located within 300 bp upstream of the FAMA gene (accessed in TAIR 9 http://gbrowse.arabidopsis.org). Expression of these small RNAs was quantitatively assayed together with FAMA and surprisingly, given the increased expression of FAMA in rdr2 plants, was positively correlated with expression of FAMA (P=0.014, R² 0.97). Upstream of SPCH (and a predited 177 bp gene At5g53205 for an unknown protein) is a cluster of rolling-curve-type helitron family transposons corresponding with 42 small RNAs and a 40 bp tandem repeat within a 427 bp dispersed repeat region. Small transposons like these are believed to be preferentially dependent on RdDM via DRM1/2 for silencing. It was hypothesised whether these small RNAs could direct non-CG methylation that spread beyond the transposable elements (TEs) to affect transcription of SPCH as has been shown for the seven tandem repeats of the F-box protein encoded by SUPPRESSOR OF drm1 drm2 cmt3 (SDC) and for RdDM arising from tandem direct repeats around the transcription start site of FWA. Expression of SPCH was measured together with expression of a subset of these siRNAs. It was found that it was inversely correlated (P=0.004, R² 0.87) so that SPCH was downregulated when expression of these siRNAs was upregulated and SPCH was methylated in LRH. On exposure to LRH stress, 24 nt siRNAs corresponding the TEs upstream of the SPCH locus were induced and assayed DNA methylation spread into the regulatory and genic regions of SPCH.

Methylation of SPCH and FAMA in isogenic progeny from single parents exposed to LRH was next examined to see whether it correlated with gene expression and SI. LRH-control progeny retained parental methylated status (FIG. 1) in the coding and non-coding regions for SPCH and exhibited similarly suppressed expression. It was therefore concluded that methylation of the SPCH locus was heritable. Conversely, the methylated status of SPCH was lost in LRH-LRH progeny, such that this gene became both unmethylated and hyper-expressed (FIG. 1). Drm1/2 progeny (LRH-control) remained unmethylated (Table 2). Unlike the WT, cmt3 LRH-control progeny were not methylated at SPCH, but methylation was induced by LRH regardless of parentage (cmt3 LRH-LRH and cmt3 control-LRH plants).

There are several plausible causes for the loss of methylation at SPCH in LRH-LRH plants, including loss of RdDM. In equal quantities of total RNA from progenies, the entire complement of small RNA duplexes was reduced in LRH-LRH plants (FIG. 3) and expression of the upstream siRNAs for SPCH was reduced (FIG. 3). This implied that heritable retention of LRH-induced methylation influenced RdDM so that it could not proceed under a repeated stress, consistent with the inability of cmt3 plants to transmit methylation to their progeny. As transgenerational DNA methylation and siRNAs were not lost in the control environment, the subsequent imposition of LRH stress must have been the causative factor rather than genomic re-setting and reactivation of methylation during the parental reproductive phase.

TABLE 2
Methytlated regions (in numbers of base pairs) of methyltransferase mutants in
control and low relative humidity (LRH) in and around the SPCH locus. ✓ indicates
positive amplification by real-time PCR of the methylated portion of genomic
DNA from each sample in comparison with the unmethylated portion.

| | 547 | 48 | 1.403 | 220 | 469 | 176 | 1.261 | 51 | 700 | 200 |
|---|---|---|---|---|---|---|---|---|---|---|
| Chr5: 21589259← | bp | bp | kb | bp | bp | bp | kb | bp | bp | bp |
| Control.met1 | — | — | — | — | — | — | — | — | — | ✓ |
| LRH.met1 | — | — | — | ✓ | — | ✓ | — | — | — | — |
| Control-control.met1 | — | — | — | — | — | — | — | — | — | ✓ |
| LRH-control.met1 | — | — | — | ✓ | — | — | — | — | — | ✓ |
| Control-LRH.met1 | — | — | — | ✓ | — | ✓ | — | — | — | — |
| LRH-LRH.met1 | — | — | — | — | — | — | — | — | — | ✓ |
| Control.drm1/2 | — | — | — | — | — | — | — | — | — | — |
| LRH.drm1/2 | — | — | — | — | — | — | — | — | — | — |
| Control-control.drm1/2 | — | — | — | — | — | — | — | — | — | — |
| LRH-control.drm1/2 | — | — | — | — | — | — | — | — | — | — |
| Control-LRH.drm1/2 | — | — | — | — | — | — | — | — | — | — |
| LRH-LRH.drm1/2 | — | — | — | — | — | — | — | — | — | — |
| Control.cmt3 | — | — | — | ✓ | — | — | — | — | — | — |
| LRH.cmt3 | ✓ | — | ✓ | ✓ | ✓ | ✓ | ✓ | — | ✓ | ✓ |
| Control-control.cmt3 | — | — | — | ✓ | — | — | — | — | — | — |
| LRH-control.cmt3 | — | — | — | ✓ | — | — | — | — | — | — |
| Control-LRH.cmt3 | ✓ | — | ✓ | ✓ | ✓ | ✓ | ✓ | — | ✓ | ✓ |
| LRH-LRH.cmt3 | ✓ | — | ✓ | ✓ | ✓ | ✓ | ✓ | — | ✓ | ✓ |

Here, loss of siRNAs could trigger active demethylation. Loss of inherited methylation at SPCH in LRH-LRH plants and in the cmt3 mutants (LRH-control) was apparent at symmetric sequences (FIG. 1). Evidence from successive generations of met1 suggests that maintenance of methylated CGs is required for the expression of Arabidopsis cytosine demethylases and that demethylated CGs are subsequently protected from de novo remethylation involving DRM2. In the whole seedlings, transcripts of the demethylating DNA glycosylase REPRESSOR OF SILENCING 1 (ROS1) were abolished by LRH in WT plants and were not detectable in LRH-LRH plants, where CG methylation of SPCH had been abolished, but were expressed in transgenerationally methylated progeny (0.43× control, P=<0.001). Expression of the demethylase DEMETER (DME) was likewise abolished by LRH in WT plants and reduced in methylated progeny of LRH parents (0.74× control) but greatly increased in LRH-LRH (50.3× control). Expression of both demethylases was undetectable in met1 control plants but was increased in met1 by LRH treatment relative to the WT (ROS 1 10.3× and DME 5.7× control). These data suggest a role for demethylation in orchestrating dynamic patterns of CG methylation in response to stress. LRH induces CG methylation but, where this is not replicated and maintained, demethylase is induced perhaps in order to protect against stochastic, de novo genome-wide asymmetric methylation and increased phenotypic abnormalities. At the SPCH locus, loss of CG methylation did not cause an increase in asymmetric methylation (FIG. 1) but there was a decrease in siRNA transcripts associated with loss of methylated CG in SPCH, as has been shown previously for the FWA locus in met1. DME is responsible for the active demethylation of maternal alleles in Arabidopsis imprinted genes and, in developing seed, its expression in the central cell causes differential methylation in the embryo and endosperm genomes. DME-mediated differential tissue demethylation during seed development is associated with activation and suppression of TEs (including a helitron TE remnant), differential siRNA accumulation and distribution, and methylation of genes in close proximity. The findings presented herein strongly suggest a similar mechanism operates on inherited methylation at the SPCH locus in stomatal precursor cells. It is not possible to rule out that ROS1 was also involved in active demethylation of transgenerational, methylated CG at an early stage, which could explain why it was undetectable in LRH-LRH seedlings when SPCH was already demethylated. It is noted, however, that ROS 1 may be less suitable than other DME-family enzymes for this type of rapid, processive demethylation. As rdr2 and dcl3 mutants do not germinate in LRH, it is likely that TE-associated siRNA accumulation and subsequent RdDM is required for correct development of the seed under stress.

Heritable methylation of SPCH was lost in progeny of transgenerationally methylated parents (LRH-control-control) (FIG. 1) and regained under LRH stress in progeny of demethylated parents (LRH-LRH-control) (FIG. 1). Predictability of the entire target SPCH gene fragment (differentially methylated in first generation plants) remained high through three generations (85%, 95% and 80% respectively). The methylation status of one symmetric site downstream of a sequence repeat at the transcription start site of SPCH explained with high fidelity the observed treatment*parent methylation pattern of the gene and its expression. The repeat sequence itself was hypomethylated in all sample plants regardless of parentage or treatment (FIG. 1). The CpG was invariably unmethylated in control plants, methylated in LRH and transgenerationally methylated in LRH-control progeny in all examined samples and amplicons (FIG. 1). At this base, predictability of demethylation in LRH-LRH was reduced to 71% and of loss and gain of methylation in the third generation reduced further to 55% in LRH-control-control and 43% in LRH-LRH-control. The decreasing predictability of intergenerational LRH-induced methylation at this site was therefore associated with the process of demethylation. It is likely that the process of demethylating and re-establishing methylation under stress following its inheritance at SPCH causes cohorts of nuclei at different stages that, therefore, have different methylation status at reproduction (mitotic and meiotic). An alternative explanation is that cytosines are protected from re-methylation following demethylation, as has been proposed previously, but that this protection is imperfect.

In contrast with SPCH, in FAMA the differential methylation pattern exhibited by parental plants was replicated in the progenies so that FAMA was methylated in all LRH plants (FIG. 1). There was evidence for inherited methylation of FAMA in a small minority (5%) of the LRH-control offspring but this was less predictable. Differential methylation of FAMA was primarily determined by growing conditions experienced by plants during development and, unlike at SPCH, was only weakly heritable. Genic CG methylation was present in LRH-LRH, relieved in LRH-control plants relative to first generation WT controls and absent in rdr6 (but not rdr2) (FIG. 1). This methylation may have been the hallmark of an older PTGS event that, unusually, was released during meiosis. Assayed siRNAs were still present in LRH-control offspring and positively correlated with FAMA expression (P=0.035, R²=0.80) except for transcripts of the genic 21 nt siRNA which were now inversely correlated with FAMA mRNA (P=0.006, R² 0.98). The experiments suggest that stress-inducible RdDM followed by meiosis may play a role in this release of genic methylation. As in tobacco, presence of PTGS-derived genic methylation did not affect FAMA expression and nor did its release. In all treatment*parent conditions, FAMA expression was highly correlated with expression of SPCH and final SI. This can be explained by interaction between FAMA and other genes upstream in the stomatal pathway which drive subsequent divisions in a dosage-dependent manner including the ICE1.SCRM2 heterodimer partners of SPCH and FAMA and SPCH itself. Increased expression of FAMA in LRH-LRH plants may have been driven largely by increased expression of SPCH, rather than by de novo methylation of FAMA, consistent with the derepression of SPCH after loss of CG methylation and the decrease in the siRNA complement. Two stress-associated kinases (MKK7 and MKK9) in the mitogen-activated protein kinase signalling cascade that interacts with the SPCH→FAMA stomatal development pathway have also been shown to have opposite effects dependent on whether they are driven by the SPCH promoter at the lineage-defining stage or by the FAMA promoter in guard mother cells. Usual inhibitors of stomatal development can be induced to promote stomata-forming divisions. The data presented herein provides evidence that, once committed to the stomatal lineage at SPCH, in planta, the majority of meristemoids will form functional guard cells and this is because of co-ordinated expression of, at least, SPCH and FAMA. Although gₛ of LRH-LRH plants was reduced when SI was increased, no more aberrant stomatal tumours, malformed precursors or clusters of stomata were noted in these plants than in the WT. SPCH additionally regulates expression of several genes in the stomatal patterning pathway and is the substrate for phosphorylation by MPK3 and MPK6 at the end of the YDA-directed MAPK signalling cascade. SPCH therefore appears to be an important hub for co-ordinating developmental and environmental cues that is itself responsive to environmental stress through RdDM.

It is proposed that the subtle interplay of both de novo and inherited methylation and demethylation at SPCH effectively “immunised” the progeny against the same stress on stomatal development experienced by their parents. This can be explained as a transgenerational “adaptive imprinting” response that is mediated by targeted DNA methylation.

LRH-LRH and LRH-control progenies apparently benefited in terms of increased biomass and seed production (FIG. 2). Fitness profiles of these progenies were altered according to the experience of their parents. A. thaliana is a self-fertilizing, annual species that will typically harbour very little genetic variation within its populations when compared with inter-populational variation. Locally, populations must therefore rely heavily on plastic resilience to accommodate fluctuations in growing conditions. Viewed in this context, an ability to moderate default physiological responses in the light of parental experiences could have considerable advantages for inbred populations to mitigate the absence of local genetic variability. It is expected to apply most strongly amongst inbreeding or apomictic perennials, where individuals suffer recurrent exposure to environmental fluctuations over many seasons.

### Methods—Summary

Supplied Arabidopsis thaliana (L.) Heynh Landsberg erecta seeds were grown in low (45%) and control (65%) relative humidity (RH) growth chambers and seed collected. Collected seeds from individual parents in both treatments were grown in low and control RH alongside untreated seeds and supplied seeds for several known methyltransferase mutants. This experiment was repeated four times; supplied seeds for known RNAi mutants were grown in the fourth repeated experiment. Each time, stomatal density (stomata mm⁻²) and index (percentage of epidermal cells forming stomata) were assessed at the same stage of growth by microscopic examination of impressions of the abaxial leaf surface. Plant dry weight, seed weight and seed number were assessed following senescence. Differential methylation with treatment and parentage was screened by high resolution melt (HRM) analysis of PCR products from known genes in the stomatal formation pathway, following bisulfite conversion of sample DNA. Full lengths and upstream of target genes were analysed for differential methylation by capturing the methylated portion of the sample genome and performing qPCR of resulting DNA for 300 bp fragments of the genes of interest. Single base-resolution methylation profiles were confirmed by bisulfite sequencing of ≧32 cloned PCR fragments for target gene regions studied. SPCH and FAMA expression levels were measured in seedling RNA by multiplexed-tandem qPCR (MT-qPCR). MT-qPCR data were analysed in comparison with housekeeping genes of equal efficiencies to target genes by two standard curve analysis. Multiple siRNAs expression was analysed by in solution hybridization and RNase digestion of the enriched small RNA fraction with custom synthesized probes, followed by electrophoretic separation and quantification of the protected probes.

### Methods—Plants and Growth Environment.

Seeds of Arabidopsis thaliana (L.) Heynh. ecotypes Landsberg erecta (Ler ref. NW20) and Columbia (Col-0 ref. N1092), methyltransferase mutants for MET1 ((Decreased Methylation 2DNA, met1 ref. N854300), Chromomethylase (cmt3 ref. N6365) and Domains rearranged methyltransferase 1/2 (drm1/drm2 ref. N6366) and for RNAi mutants RNA dependent rna polymerase 2 (rdr2 ref. N850602), RNA dependent rna polymerase 6 (rdr6 ref. N24285), Dicer-like 3 (dcl3 ref. N505512) and Dicer-like 4 (dc14 ref N6954) were supplied by NASC (Nottingham, UK). Seeds were sown in seedling compost (Sinclair, Lincoln, U.K.), germinated and grown in controlled environment growth cabinets (Saxcil, R. K. Saxton, Bredbury, Cheshire, U.K.) until harvest, according to ARBC guidelines except that the relative humidity of one cabinet was controlled at 45%±5 whilst the other was maintained at 65%±5. After 64 d, stage 9.70, seeds were harvested from each individual. Harvested Ler seeds, supplied Ler seeds (as before) and supplied seeds for mutants (as before) were sown, germinated and grown as before except that growth cabinets were swapped and no stratification was applied. Different (rotated) growth chambers were used in each of the 4 repeated experiments to accommodate for growth chamber effects (Sanyo Gallenkamp, Loughborough, U.K.). The complete dry biomass and seed mass of individual harvested plants were weighed and seeds counted, following threshing through a series of graded meshes, by capturing a digital image of collected seeds using an Epson Perfection 3170 scanner (Epson (U.K.), Hemel Hempstead, U.K.) then particle analysis using ImageJ software version 1.37 (freeware NIH, USA).

### Methods—Stomatal Analyses

Stomatal density (stomata mm⁻²) and index (percentage of epidermal cells forming stomata) were determined by making impressions of the entire abaxial surface of one mature rosette leaf (insertion 6-8, length approximately 40 mm) and one cauline leaf (insertion 13-15, length approximately 15 mm) from 48 plants (each of 16 replicate plants from each of 3 individual parents in the treatment*parent experiments) at the same physiological stage (6.50) in each repeated experiment. Digital images were then captured from an Axioscope 2 microscope with an Axiocam camera attached (Carl Zeiss Ltd), using Axio Vision 3.1 (Image Associates, Oxfordshire, UK) software and the number of stomata and other epidermal cells per unit area counted using ImageJ software (as before). Gas exchange (stomatal conductance to water vapour and instantaneous leaf-level water use efficiency) was measured using the Lcₚᵣₒ₊ infra-red gas analyzer with Arabidopsis leaf chamber (ADC BioScientific, Great Amwell, U.K.) in 6 replicate plants pre-conditioned in ambient RH in the dark for 12 hr.

### Methods—DNA Methylation Analyses

Whole seedlings (first true leaf stage), mature and immature leaves from ≧12 replicate plants were snap frozen in liquid nitrogen and stored at −80° C. DNA was extracted using the Dneasy plant mini-kit (Qiagen, U.K.) according to the manufacturer's instructions. 2 μg genomic DNA was then modified by bisulfite treatment using the EZ DNA methylation kit (Zymo Research, Orange, Calif.) according to the manufacturer's instructions. Desulphonated DNA was diluted 1 in 5. High resolution melt (HRM) analysis was used to analyse differential methylation with treatment as in Wojdacz, T. K. & Dobrovic, A. Methylation-sensitive high resolution melting (MS-HRM): a new approach for sensitive and high-throughput assessment of methylation. Nucleic Acids Res. 35, No. 6 e41 (the content of which is incorporated herein by reference in its entirety) except that each 20 μl reaction mix contained 1× Biomix (Bioline, London, U.K.), 25 μM Syto9 dye (Invitrogen, Carlsbad, Calif.) and 300 nM each forward and reverse bisulfite-specific primer for the gene of interest. PCR amplification conditions used were: 2 min at 95° C., then 50 cycles of 95° C. for 15 s and 50° C. for 30s, 60° C. hold for 1 min and HRM from 58-80° C. at 0.5° C. s⁻¹. For each gene, untreated genomic DNA (diluted 1 in 1000) was included as a positive control using the equivalent (but not bisulfite-specific) primer. Differential methylation with treatment was identified using the RotorGene™ 6000 Series Software version 1.7 (Corbett Research UK Ltd., Cambs., U.K.) at an 80% confidence level. Assays were repeated 6-8 times for genes putatively identified as differentially methylated.

Positive results indicating differential methylation in the SPCH and FAMA genes were validated by capturing the methylated portion of genomic DNA using the Methylamp Methylated DNA Capture kit (Epigentek, Cambridge Bioscience, Cambridge, U.K.) and performing comparative qPCR analysis using negative controls provided in the kit (Ig mouse antibody). Subsequently, primers were designed to target every 300 bp of the coding regions, for 600 bp of (5′) upstream regions of the SPCH and FAMA genes and for the 2.3 kb upstream genomic region of SPCH. qPCR and HRM conditions were as described above except that 15 ng of template DNA were used, Tₐ was 56° C. and an extension phase of 66° C. for 6 min replaced the 1 min hold; HRM was from 68-90° C.

Base-pair resolution methylation profiles were obtained by sequencing ≧32 cloned amplicons (vector pCR2.1; Invitrogen, Carlsbad, Calif.) per sample of three, pooled replicate plants (Geneservice, Source Bioscience PLC, Nottingham, U.K.) following bisulfite treatment and PCR, as described above except that 5 nM labelled, synthetic DNA with methylated and unmethylated cytosines for each PCR product (Sigma-Aldrich Ltd., Gillingham, U.K.) was added to the 2 μg sample DNA prior to bisulfite treatment as a positive control for complete bisulfite conversion. Differential methylation was assessed with reference to the unmodified genomic DNA sequence and comparison of cytosine to thymine conversion between treatments. Sequences were aligned using ClustalW2 and predictability calculated as the inverse of entropy using BioEdit v. 7.0.9.0.

Following each round of FIRM, qPCR and PCR, a sample of products was analysed for size accuracy and purity using the Agilent Bioanalyzer Series II DNA 1000 chip (Agilent, Winnersh, U.K.).

### Methods—RNA Expression Analyses

Total RNA was isolated from frozen leaf material using the RNeasy Plant Mini kit (Qiagen, U.K.) according to the manufacturer's instructions. Primers for Multiplexed Tandem PCR (MT-PCR) were designed for the target genes SPCH and FAMA and for the internal control genes PP2A and SAND. MT-PCR was performed as in Stanley, K.K. & Szewczuk, E. Multiplexed tandem PCR: gene profiling from small amounts of RNA using SYBR green detection. Nucleic Acids Res. 33, 20 e180 (2005) (the content of which is incorporated herein by reference in its entirety) using 500 ng starting RNA, except that Sensimix (Quantace, London, U.K.) reverse transcriptase and buffer were used, and reverse transcription was executed at 45° C. for 15 min followed by 70° C. for 15 min. First round multiplexed amplification was carried out in the ABI9700 thermal cycler using Sybr Premix Ex Taq polymerase (Takara Bio Europe, Saint-Germain-en-Laye, France) and final volumes of 200 nM for each primer. PCR was performed under the following conditions: 1 min at 95° C., 10-15 cycles of 95° C. for 15 s, 58° C. for 20 s and 72° C. for 15 s then 72° C. for 7 min. Pre-amplification products were diluted 1:1 and second-round PCRs prepared with Sybr Premix Ex Taq (as before) and internal primers and 1 μA template cDNA. qPCR was carried out in the RotorGene™ 6000 thermal cycler (Corbett Research UK Ltd., Cambs., U.K.) using the following conditions: 95° C. for 1 min, then 40 cycles of 95° C. for 10 s, 60° C. for 20 s and 72° C. for 8 s and HRM from 70-96° C. at 0.5° C. s⁻¹. All reactions were prepared in triplicate and serial dilutions completed for genes of interest and controls. RotorGene™ 6000 Series software version 1.7 was used to determine gene amplification efficiencies and RNA quantification (as before) employing the two standard curve method.

Following each round of PCR and qPCR, a sample of products was analysed for size accuracy and purity using the Agilent Bioanalyzer DNA 1000 chip and kit (as before).

Methods—siRNA Analyses

Total RNA was isolated from seedling samples using the mirVana miRNA isolation kit (Ambion, Warrington, U.K.) according to the manufacturer's instructions, checked and quantified using the Agilent Bioanalyzer RNA 6000 Nano and Small RNA chips and kits (Agilent, Winnersh, U.K.) against small dsRNA standards (New England Biolabs, Hitchin, U.K.). 200 ng total RNA from each sample was enriched for the small RNA fraction using the isolation kit (as before). Unlabelled antisense RNA probes of differing nt lengths were designed and constructed using the mirVana probe construction kit (Ambion, Warrington, U.K.) for SPCH, FAMA and local smRNAs; <four probes were detected in each reaction using the mirVana detection kit (Ambion, Warrington, U.K.) according to the manufacturer's instructions. Probes were post-labelled and visualised fluorescently using the Agilent Bioanalyzer Small RNA chip (as before) and small dsRNA standards ladder (as before).

### Methods—Primer Designs

Primer designs for DNA methylation, RNA and siRNA analyses are included as Tables 1, and 3-5. All primers were designed using Primer3 software; bisulfate-specific primers were based on the returned, bisulfite-specific sequence from MethPrimer software.

TABLE 3
Primers for unmodified genomic DNA (equivalent to bisulfite-specific
region assayed). These primers were also used for qPCR following
capture of methylated DNA.

| Gene | GenBank | Primer | |
|---|---|---|---|
| name | accession no. | name | DNA sequence (5′-3′) |
| FAMA | At3g24140 | FAMAFu | CAAACTTCTTAGGTGAATCCTCAGG (SEQ ID NO: 43) |
| FAMA | At3g24140 | FAMARu | ATAGTTTTCCACAACGAGGTTTAGG (SEQ ID NO: 44) |
| FAMA | At3g24140 | FAMA2Fu | CTTCTTCTACTATCTTGCATGTCTTG (SEQ ID NO: 45) |
| FAMA | At3g24140 | FAMA2Ru | AAACTTCTTAGGTGAATCCTCAGG (SEQ ID NO: 46) |
| FAMA | At3324140 | FAMAFup | TCTTCAAAAAATTGACCATT (SEQ ID NO: 47) |
| FAMA | At3g24140 | FAMARup | AACTGCATGCTCTCTTTCTAA (SEQ ID NO: 48) |
| SPCH | At5g53210 | SPCHFu | CCAATCTTTGGAAGCCAAGAAACAA (SEQ ID NO: 49) |
| SPCH | At5g53210 | SPCHRu | GTTGACCGGTTACTCGACATTCTCG (SEQ ID NO: 50) |
| SPCH | At5g53210 | SPCHFup | CCATCTTAGAGAGTCTTGAAGGTGC (SEQ ID NO: 51) |
| SPCH | At5g53210 | SPCHRup | CAACGGGTAGAACGAATAATAGAAGAAGAC (SEQ ID NO: 52) |

TABLE 4
Primers for qPCR of methylated genomic DNA following enzymatic
capture and amplification of DNA for 454 sequencing.

| Gene | GenBank | Primer | |
|---|---|---|---|
| name | accession no. | name | DNA sequence (5′-3′) |
| FAMA | At3g24140 | FAMAF1 | AAAGCAATCGATGCCACAAC (SEQ ID NO: 53) |
| FAMA | At3g24140 | FAMAR1 | AGTCCGCAAACTGCATCAC (SEQ ID NO: 54) |
| FAMA | At3g24140 | FAMAF2 | CATCACTACCATGGAACAAACC (SEQ ID NO: 55) |
| FAMA | At3g24140 | FAMAR2 | CTGTTGGATGGAACTTGCTATG (SEQ ID NO: 56) |
| FAMA | At3g24140 | FAMAF3 | GCTCATTATTACGGGAAATGTAA (SEQ ID NO: 57) |
| FAMA | At3g24140 | FAMAR3 | CCCGGCCTTCTTCTTGAAA (SEQ ID NO: 58) |
| FAMA | At3g24140 | FAMAF4 | GAAACGAGGTTTACGGCAGA (SEQ ID NO: 59) |
| FAMA | At3g24140 | FAMAR4 | GGGACCAACAGAAACTTATCAAA SEQ ID NO: 60) |
| FAMA | At3g24140 | FAMAF5 | AAAAGATATTGGTGGTTCGATG (SEQ ID NO: 61) |
| FAMA | At3g24140 | FAMAR5 | AAGCAAATAATCATAACATCTAAAAGG (SEQ ID NO: 62) |
| FAMA | At3g24140 | FAMAF6 | CTCCAAGCATTTGGAAGAGTG (SEQ ID NO: 63) |
| FAMA | At3g24140 | FAMAR6 | CCGCTTGTTCAAAACCTACAA (SEQ ID NO: 64) |
| SPCH | At5g53210 | SPCHF1 | CCATCTTAGAGAGTCTTGAAGGTGC (SEQ ID NO: 65) |
| SPCH | At5g53210 | SPCHR1 | CAACGGGTAGAACGAATAATAGAAGAAGAC (SEQ ID NO: 66) |
| SPCH | At5g53210 | SPCHF2 | GTTTCTGGTAGTGCCCGACT (SEQ ID NO: 67) |
| SPCH | At5g53210 | SPCHR2 | CATAGATATGCATGATACTTTTGATGT (SEQ ID NO: 68) |
| SPCH | At5g53210 | SPCHF3 | TCCAATCTTTGGAAGCCAAG (SEQ ID NO: 69) |
| SPCH | At5g53210 | SPCHR3 | GGCTAAGAGGCGGTTTTCTT (SEQ ID NO: 70) |
| SPCH | At5g53210 | SPCHF4 | AACCACCACCAGATTCACCA (SEQ ID NO: 71) |
| SPCH | At5g53210 | SPCHR4 | GAGTGGTAGTTGCGGTGGAA (SEQ ID NO: 72) |
| SPCH | At5g53210 | SPCHF5 | TCATCCTAATTAATTTTCACTGACTTG (SEQ ID NO: 73) |
| SPCH | At5g53210 | SPCHR5 | TTCTTCCCCCACCATATATCC (SEQ ID NO: 74) |
| SPCH | At5g53210 | SPCHFmet | CAACTGGCCAATGAGCTGTA (SEQ ID NO: 75) |
| SPCH | At5g53210 | SPCHRmet | AAGTCCTCGAACACCTCAGC (SEQ ID NO: 76) |
| SPCH | At5g53210 | SPCHFumet | AATATTAACACCGTCGACGAAA (SEQ ID NO: 77) |
| SPCH | At5g53210 | SPCHRumet | GCTGAATTTGTTGAGCCAGTT (SEQ ID NO: 78) |
| SPCH | At5g53210 | SPCH6F | GAAGAGCCCCCAAAATCTTC (SEQ ID NO: 79) |
| SPCH | At5g53210 | SPCH6R | TCCCTACTTGATCTCTGATCTTGTT (SEQ ID NO: 80) |
| SPCH | At5g53210 | SPCH7F | TTTTCGTTGGGAGTTTAGTGC (SEQ ID NO: 81) |
| SPCH | At5g53210 | SPCH7R | TGTTGAGCCAGTTCTTCTGC (SEQ ID NO: 82) |
| SPCH | At5g53210 | Up1F | CGCTATACAACGAATCCATGA (SEQ ID NO: 83) |
| SPCH | At5g53210 | Up1R | TCACGGGATGGGTAAAGAAA (SEQ ID NO: 84) |
| SPCH | At5g53210 | Up2F | CTAATGAACGGACGGTTTGC (SEQ ID NO: 85) |
| SPCH | At5g53210 | Up2R | TGGGCTAAAATAATTGGGACA (SEQ ID NO: 86) |
| SPCH | At5g53210 | 53205F | TGCGTTAGGACTATCCATTTCA (SEQ ID NO: 87) |
| SPCH | At5g53210 | 53205R | ATGCAACATCGAATCATCCA (SEQ ID NO: 88) |
| SPCH | At5g53210 | Up3F | TCCATACTTTCACCCAAAAAGAA (SEQ ID NO: 89) |
| SPCH | At5g53210 | Up3R | TCTTGCAACACAAAATGTTAAGG (SEQ ID NO: 90) |
| SPCH | At5g53210 | Up4F | TTTAAACTCCATATCTTTGCAGAAAAC (SEQ ID NO: 91) |
| SPCH | At5g53210 | Up4R | TTCGTATAAACCTTAACGAGAGAGC (SEQ ID NO: 92) |
| SPCH | At5g53210 | Up1AF | CCCATCCCGTGATTTATTTTT (SEQ ID NO: 93) |
| SPCH | At5g53210 | Up1AR | TTACCCAACCATTTTTGCAC (SEQ ID NO: 94) |
| SPCH | At5g53210 | SPCH1AF | TTTCTCCGGTTACGTTCCAC (SEQ ID NO: 95) |
| SPCH | At5g53210 | SPCH1AR | TCCGACAGCTGCATCTACAC (SEQ ID NO: 96) |

TABLE 5
Primers for MT-qPCR of stomatal developmental and endogenous control
genes Primers for qPCR of ROS1 and DME were as designed and used by¹⁵.
Primers for qPCR of RDR2 were RDR2F (5′-GGGTCCAGAGCTTGAGACTG-3′ (SEQ
ID NO: 113)) and RDR2R (5′- CCCTTCTCCAAGGATTGACA-3′(SEQ ID NO: 114)).
Primers for qPCR of DCL3-1 were DCL3F (5′-GTCTTTGAGCCGTTGCTTTC-3′ (SEQ
ID NO: 115)) and DCL3R (5′- GTGAAGCTGCTTTTCCCAAG-3′(SEQ ID NO: 116)).
Primers for genotyping methyltransferase mutants were as in²³⁻²⁵, flank-
ing the insertion AAGTGGCACTTCATCGTCTCCCAATCAAAATGAAGCT (SEQ ID NO: 117)
(GenBank accession CC887813) for DRM2. Antisense probes for siRNA analyses
were designed to the small RNA sequences downloaded from the Arabidopsis
Small RNA Project database (http://asrp.cgrb.oregonstate.edu/)^{19,36-40} for
the region 3: 8714.3k . . . 8721k for FAMA and 5: 21601k . . . 21611.4k
for SPCH. In this database SPCH is currently located at 5: 21603.8k.
Additional (A) bases were added to artificially create different length
probes The antisense probe for FAMA RNA was CUUCUGCCGUAAACCUCGUUUCACUUGaaaa
(SEQ ID NO: 118) and for SPCH was UUAAGUGCUCGUUCAUUUGCUUUCUCCGaaaa
(SEQ ID NO: 119).

| Gene | GenBank. | Primer | |
|---|---|---|---|
| name | accession no. | name | DNA sequence (5′-3′) |
| FAMA | At3g24140 | FAMA EF | CAAGTGAAACGAGGTTACGG (SEQ ID NO: 97) |
| FAMA | At3g24140 | FAMA ER | GTACAAAGTTCTCGCCGTGT (SEQ ID NO: 98) |
| FAMA | At3g24140 | FAMA IF | TTTACGGCAGAAGACATAGCAAA (SEQ ID NO: 99) |
| FAMA | At3g24140 | FAMAIR | TCATCACATTGTCAATAGATTGGAG (SEQ ID NO: 100) |
| SPCH | At5g53210 | SPEEC EF | TGGAACGTAACCGGAGAAAG (SEQ ID NO: 101) |
| SPCH | At5g53210 | SPEEC ER | ACGTTGTTTCTTGGCTTCCA (SEQ ID NO: 102) |
| SPCH | At5g53210 | SPEEC IF | CGGAGAAAGCAAATGAACGA (SEQ ID NO: 103) |
| SPCH | At5g53210 | SPEEC IR | CCACAACTCCTCCTATGATCG (SEQ ID NO: 104) |
| PP2A | At1g13320 | PP2A EF | TCCGAGATCACATGTTCCAA (SEQ ID NO: 105) |
| PP2A | At1g13320 | PP2A ER | TCATCACATTGTCAATAGATTGGAG (SEQ ID NO: 106) |
| PP2A | At1g13320 | PP2A IF | ATTCCGATAGTCGACCAAGC (SEQ ID NO: 107) |
| PP2A | At1g13320 | PP2A IR | TGCGAAATACCGAACATCAA (SEQ ID NO: 108) |
| SAND | AT2G28390 | SAND EF | CCCGACATATCTGTGGGAAC (SEQ ID NO: 109) |
| SAND | AT2G28390 | SAND ER | TGGGGTCCCAATCCTTTTAC (SEQ ID NO: 110) |
| SAND | AT2G28390 | SAND IF | GGAATTCTCACCCCCCAGTAAC (SEQ ID NO: 111) |
| SAND | AT2G28390 | SAND IR | GGGTCCCAATCCTTTTACA (SEQ ID NO: 112) |

### Example 2

### Inherited Acquired Drought Tolerance Induced by Low Relative Humidity in Arabidopsis

When parental plants are grown under control relative humidity, the imposition of a short period of drought caused a marked and significant reduction in chlorophyll content (FIG. 4; Gen1 controlRH) and total plant dry mass (FIG. 5; Gen1 controlRH). In contrast, when the same genotypes were grown under constant low relative humidity (LRH), the imposition of the same periodic drought did cause a fall in chlorophyll content relative to the undroughted controls (FIG. 4; Gen1 LRH) and surprisingly was associated with an increase in final dry mass (FIG. 5; Gen1 controlRH). Offspring generated from seed collected from plants exposed to control relative humidity behaved similarly in control conditions, with a marked drop in chlorophyll content (FIG. 4; Control-Control) and final dry Biomass (FIG. 5; Control-Control). Remarkably, offspring generated from seed collected from parent plants exposed to low relative humidity showed a significant increase in chlorophyll content when exposed to the same periodic drought (FIG. 4; LRH-Control) and a similar significant increase in total dry biomass (FIG. 5; LRH-Control). Thus, the parental exposure to LRH positively changed the response of the offspring to periodic drought. Even more remarkably, offspring from parental plants grown under low relative humidity when exposed to the same levels of low relative humidity as their parents, they had become insensitive to periodic drought in terms of chlorophyll content (FIG. 4; LRH-LRH) and dry biomass (FIG. 5; LRH-LRH). Unexpectedly, the effect of low RH pre-conditioning from the first generation was not heritable into a third generation, when grown under control conditions in terms of chlorophyll content (FIG. 4; LRH-control-control) and biomass (FIG. 5; LRH-control-control). Third generation offspring exposed to two successive LRH conditions followed by a control with and without drought behaved essentially similar to LRH-control treatments (FIGS. 4 and 5). Mutants for de novo methylation (drm1/2) and (redundantly) the maintenance of methylation (cmt3) were similarly affected by drought in control RH but only the drm1/2 mutant was rescued by low RH pre-conditioning. Thus, the exposure of parental plants to variable relative humidity conditions incurred a changed epigenetic response to periodic drought in the same and following generations.

### Methods—Plants and Growth Environment.

Seeds of Arabidopsis thaliana (L.) Heynh. ecotypes Landsberg erecta (Ler ref. NW20), Chromomethylase (cmt3 ref. N6365) and Domains rearranged methyltransferase 1/2 (drm1/drm2 ref. N6366) were supplied by NASC (Nottingham, UK). Seeds were sown in seedling compost (Sinclair, Lincoln, U.K.), germinated and grown in controlled environment growth cabinets (Saxcil, R. K. Saxton, Bredbury, Cheshire, U.K.) until harvest, according to ARBC guidelines except that the relative humidity of one cabinet was controlled at 45%±5 whilst the other was maintained at 65%±5. Harvested Ler seeds, supplied Ler seeds (as before) and supplied seeds for mutants (as before) were sown, germinated and grown under control RH before except that after 40 d half of the plants from each genotype were subjected to a drought treatment by withholding watering for 4 d. After this treatment, watering was restarted until seeds were harvested from each individual on day 64 (stage 9.70). Growth cabinets were swapped and no stratification was applied. Different (rotated) growth chambers were used in each of the 4 repeated experiments to accommodate for growth chamber effects (Sanyo Gallenkamp, Loughborough, U.K.). Each time, stomatal density (stomata mm⁻²) and index (percentage of epidermal cells forming stomata) were assessed at the same stage of growth by microscopic examination of impressions of the abaxial leaf surface (as described above). The complete dry biomass and seed mass of individual harvested plants were weighed and seeds counted, following threshing through a series of graded meshes, by capturing a digital image of collected seeds using an Epson Perfection 3170 scanner (Epson (U.K.), Hemel Hempstead, U.K.) then particle analysis using ImageJ software version 1.37 (freeware NIH, USA).

### Example 3

### Inherited Increased Resistance to Pathogen Botrytis cynerea in Arabidopsis Offspring

It has been found that Arabidopsis plants when innoculated with a pathogenic strain of Botrytis cynerea, respond in the short term by activating the expression of specific disease resistance genes. At the same time the global methylation pattern of the plant genome also changes. Quite remarkably, however, seeds collected from these plants present a higher resistance to the pathogen when innoculated. The plants used are so inbred that the offspring are to all intents and purposes genetically identical to the parent.

Second generation wild type plants were sown to compare whether any changes at methylation level are transmitted to next generation. Morphological data revealed existence of a transgenerational acquired increased resistance to Botrytis cynerea on the wild type Langsberg erecta genotype while none of the methylation mutants showed such increase in resistance (FIG. 6). Generation 2 plants were treated with Botrytis cynerea. Pictures show lesions associated to fungal infection three days after inoculation in Langsberg erecta. Offspring of non-inoculated plants (A), offspring of inoculated plants (B) Arrows point inoculated leaves. Detail of the inoculated leaves from offspring of non-inoculated plants (C), offspring of inoculated plants (D).

TABLE 6
Estimation of the resistance to B. cynerea in A. thaliana Lansberg erecta (Wild type -
Laer) and methylation mutants drm1/2 (Drm), chr1 (Chr), cmt3-7 (Cmt) and kyp2 (Kyp) three
days after inoculation.

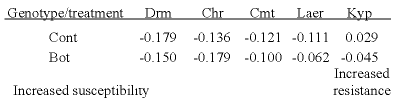

Cont: Offspring of non-inoculated plants.
Bot: offspring of inoculated plants.
Lighter shading indicates resistance to pathogen, darker shading susceptibility based on morphological analysis.

Analysis of global methylation changes induced by infection with Botrytis cynerea using MSAP (with the enzyme combination MspI/EcoRI that is sensitive to methylation on the CpHpG motif) did not show significant differences between infected and non infected plants (FIG. 7). Conversely, when the enzyme combination HpaII/EcoRI (sensitive to methylation on the CpHpG and the CpG motiffs) was used, genotypes drm1/2 and chr1 showed a significant change on global DNA methylation induced by the infection with Botrytis. Surprisingly, no differences were found between treatments for genotypes Wild type—Laer and kyp2. While genotype cmt3-7 showed some degree of separation (not significant) between samples infected with Botrytis and those non-infected (FIG. 8). Remarkably, observed changes on global DNA methylation associated to Botrytis infection present an inverse correlation with the acquired increased resistance described above. These results suggest that maintenance of DNA methylation is necessary to generate acquired resistance.

### Methods—Plants and Growth Environment.

Seeds of Arabidopsis thaliana (L.) Heynh. ecotypes Landsberg erecta (Ler ref. NW20) and mutants Chromatin-remodeling ATPase (CHR1 ref. N30937) Chromomethylase (cmt3 ref N6365) and Domains rearranged methyltransferase 1/2 (drm1/drm2 ref. N6366) and Kryptonite-2 (KYP-2 ref. N6367) were acquired from the European Arabidopsis Stock Centre.

Plants were grown in seedling compost (Sinclair, Lincoln, U.K.) in 24 cell trays with 1 plant in each 4 cm×4 cm cell, germinated and grown in controlled environment growth cabinets (Saxcil, R. K. Saxton, Bredbury, Cheshire, U.K.) until harvest, according to ARBC guidelines. One cell was removed to allow for bottom watering. Seeds were germinated at 4° C. and grown for 1 week under glass before being transferred to experimental conditions in a controlled-environment growth room. The plants were grown at 22° C. under an 8 hour photoperiod (approx. 70 μmol/m²/s) to inhibit flowering. After 64 d, stage 9.70, seeds were harvested from each individual. The complete dry biomass and seed mass of individual harvested plants were weighed and seeds counted, following threshing through a series of graded meshes, by capturing a digital image of collected seeds using an Epson Perfection 3170 scanner (Epson (U.K.), Hemel Hempstead, U.K.) then particle analysis using ImageJ software version 1.37 (freeware NIH, USA).

### Generation Zero (G0)

To homogenize and standardize the level of methylation across the plant material a Generation 0 (Five plants per genotype) was grown in standard conditions: 24° C. short days (8 h light/16 h darkness), under light intensity of 100 mol m⁻² s⁻¹. It allowed excluding any possible epigenetic variation which could exist due to variable seed storage conditions. Seeds obtained from each single plant of each genotype of Generation 0 were used in the subsequent part experiment—growing Generation one (G1). Seeds were collected from a single individual to insure the maximum level of genetic homogeneity across the plant material. Harvested Ler seeds, supplied Ler seeds and harvested mutant seeds supplied seeds for mutants. Seeds were sown, germinated and grown as before except that growth cabinets were swapped.

### Generation One (G1)

Four trays were planted (92 plants). Plant trays were randomly assigned for two different treatments (innoculation with Botrytis cynerea and control). Plants were inoculated with the necrotrophic gray mold fungus Botrytis cynerea (strain iMi 169558, International Mycological Institute, Kew, U.K.) five weeks after germination. Plants were treated with 1×10⁵ spores mL⁻¹ suspension, by placing 2 droplets directly on the upper side of leaf number five (in order to ensure that they were at the same developmental stage) using a pipette. Seven days after inoculation, leaf six was sampled from half of the plants from each treatment and sampled plants were discarded. Seeds were collected from five of the remaining individuals and pooled to obtain a significant representation of the epigenetic variability induced by the treatments. Harvested Ler seeds, supplied Ler seeds and harvested mutant seeds supplied seeds for mutants. Seeds were sown, germinated and grown as before except that growth cabinets were swapped in the subsequent part experiment—growing Generation one (G2).

### Generation One (G2)

Eight trays were planted (184 plants). Plant trays were randomly assigned for two different treatments (innoculation with Botrytis cynerea and control) as described above. Plants arising from seeds obtained from treated and untreated plants were inoculated again (see Table 6) as described above. Five days after inoculation pictures were taken from the whole plant and inoculated leaves for each of the genotype-G1-G2 treatment groups for documentation and image analysis. Susceptibility or resistance to fungal infection was assessed by measuring the size and intensity of the lesions resulting from Botrytis cynerea inoculation. Seven days after inoculation, leaf six was sampled from half of the plants from each treatment and sampled plants were discarded. Seeds were collected from five of the remaining individuals and pooled to obtain a significant representation of the epigenetic variability induced by the treatments. Generation 2 plants were looked at, specifically morphological changes associated with different background (G1treatment) within the same G2 treatment group.

TABLE 7

| Treatment/Generation | Control G1 | Botrytis G1 |
|---|---|---|
| Control G2 | CC | BC |
| Botrytis G2 | CB | BB |

### Methods—DNA Extraction

All DNA extractions were carried out using kits from Qiagen following the manufacturer's instructions. The DNeasy® plant mini kit was used for extracting DNA from A. Thaliana samples from 23 out the 46 plants per treatment. Reagents discussed below all derive from this kit.

Approximately 100 mg of plant tissue was disrupted in liquid nitrogen in a 1.5 ml microcentrifuge tube using a pair of scissors. Immediately, without allowing the tissue to thaw, 400 μl of lysis buffer AP1 preheated to 65° C. and 4 μl of RNase A were added to each tube. The contents were mixed by inversion and incubated at 65° C. for 10 min with occasional mixing every 2-3 min.

Following this, 130 μl of AP2 buffer was added to each sample and the tubes were incubated on ice for 5 min to precipitate the proteins and polysaccharides. Tubes were then subjected to centrifugation for 5 min at 13,000 rpm to precipitate viscous lysates and other solids.

The supernatant was then transferred to a QIAshredder™ column (with silica gel matrix) and centrifuged at 13,000 rpm for 2 min to remove precipitates and cell debris. The column flow-through was collected and transferred into a fresh tube and mixed with 0.5 volumes of wash buffer and 1 volume of ethanol. This mixture was transferred into a second DNeasy mini spin column and subjected to centrifugation at 8,000 rpm for 1 min. The flow-through was discarded since DNA molecules are retained on the column. The bound DNA was washed twice by passing 500 μl of wash buffer AW through the column by centrifugation at 8,000 rpm for 1 min.

Subsequently, the membrane was dried by centrifugation at 13,000 rpm for 1 min after the addition of 100 μl of buffer AE preheated to 65° C. and incubation for 5 min at room temperature.

### Methods—Quantification by Agarose Gel

Aliquots of DNA (1-5 μl) were subjected to 1% (w/v) agarose gel electrophoresis to determine quality and quantity of DNA present. The gel was prepared by dissolving of appropriated quantity of agarose in the appropriate volume of 1×TAE buffer (40 mM tris-acetate, 1 mM EDTA) followed by heating in a microwave oven until all the agarose had melted. The gel solution was cooled to ˜50° C. before adding 10 mg/ml ethidium bromide solution to a final concentration of 0.35 μg/ml and then poured into a casting tray with an appropriate comb in place to create the loading well.

When set, the gel was transferred into a horizontal electrophoresis apparatus with the gel comb at the cathode end. The gel comb was removed and sufficient 1×TAE buffer was added to the electrode chamber to cover the gel by approximately 1 mm. Prepared DNA samples (5 μl DNA: 1 μl blue loading dye [0.23% (w/v) bromophenol blue, 60 mM EDTA, 40% (w/v) sucrose]) were then loaded into the gel wells. HyperLadderII (Bioline, BIO-33040) size markers were loaded into the flanking lanes. The gels were subjected to electrophoresis at constant voltages ranging from 3-5 V/cm for 15-60 min. The DNA was visualized using a UV transilluminator (320 nm wavelength).

### Methods—Methylation-Sensitive Amplified Fragment Length Polymorphism

Methylation-Sensitive Amplified fragment length polymorphism (AFLP) was performed on a randomly selected eight DNA samples per treatment and was based on the AFLP protocol described by Vos et al (1995) but using isoschizomers targeting the same recognition motif.

The basis of the technique is the detection of restricted fragments of genomic DNA through polymase chain reaction (PCR) amplification. It allows the creation of fingerprints from DNA of any origin or complexity using a limited set of generic primers and needs no prior knowledge of sequences. The use of restriction enzymes sensitive to methylation adapts this method for detection of methylation.

The DNA was restricted with 2 restriction enzymes, one rare and one common cutter sensitive to cytosine methylation. Two different restrictions were carried out with isoschizomers of the common cutter sensitive to different types of cytosine methylation. All enzymes were obtained from Fermentas, Canada.

MspI enzyme: Cuts between the two cytosines of the sequence 5′CCGG 3′ and its action is prevented by methylation on the first C but not by methylation on the second C.

HpaII enzyme: Cuts between the two cytosines of the sequence 5′CCGG 3′ and its action is prevented by methylation on the second C but not by methylation on the first C.

TABLE 8
Restriction reactions

| MspI/EcoRI | | | HpaII/EcoRI | | |
|---|---|---|---|---|---|
| DNA | 10-25 | μl | DNA (300 ng) | 10-25 | μl |
| (300 ng) | | | | | |
| Tango buffer | 7 | μl | Tango buffer | 3 | μl |
| (10X) | | | (10X) | | |
| MspI | 2 | μl | HpaII (10 u/μl) | 1 | μl |
| (10 u/μl) | | | | | |
| EcoRI | 1 | μl | Water | to make up 30 | μl |
| (10 u/μl) | | | | | |

| | | | |
|---|---|---|---|
| Water | to make up 35 | μl | Incubated at 37° C. for 2 h |
| | | | before adding: |

| | | | |
|---|---|---|---|
| Incubated at 37° C. for 3 h | EcoRI (10 u/μl) | 1 | μl |
| | Tango buffer | 3.75 | μl |
| | (10X) | | |

Incubated at 37° C. for a further 1 h

Adaptors specific to the restriction sites are ligated onto the DNA to allow for the amplification of fragments with generic primers and without the need for sequence information to be obtained first. All enzymes were from Fermentas and the adaptors were from Sigma-Genosys Ltd.

TABLE 9
Adaptor structure

| | EcoRI | MspI/HpaII |
|---|---|---|
| Forward | 5′ CTCGTAGACTGCGTACC | 5′ GACGATGAGTCTCGAT |
| | 3′ | 3′ |
| | (SEQ ID NO: 120) | (SEQ ID NO: 121) |
| Reverse | 3′ CTGACGCATGGTTAA 5′ | 3′ TACTCAGAGCTAGC 5′ |
| | (SEQ ID NO: 122) | (SEQ ID NO: 123) |

An adaptor mix was created by combining 1 nM of the EcoRI adaptor and 10 nM of the MspI/HpaII adaptor.

TABLE 10
Ligation reaction

| | |
|---|---|
| Digested DNA | 35 μl |
| Ligation buffer (10X) | 5 μl |
| T4 ligase (1 u/μl) | 1.4 μl |
| Adaptor mix | 5 μl |
| Water | 3.6 μl |

Incubated at room temperature overnight.

The amplification rounds were carried out using one oligonucleotide primer that corresponded to the EcoRI ends and one oligonucleotide primer that corresponded to the MspI/HpaII ends. The first round of amplification reduces the number of possible fragments by the addition of one extra base at the 3′ end of the primer, while the second round of amplification further reduces the amount of possible fragments by the addition of one or two addition bases at the 3′ end of the primer. The second round EcoRI primers were labelled 6-Fam (Carboxyfluorescein) to allow visualisation of the products.

TABLE 11
Pre-amplification primers

| EcoRI | MspI/HpaII |
|---|---|
| 5′ AGACTGCGTACCAATTCA 3′ | 5′ GATGAGTCTCGATCGGA 3′ |
| (SEQ ID NO: 124) | (SEQ ID NO: 125) |

TABLE 12
Pre-amplification PCR mix

| | |
|---|---|
| Restricted + ligated DNA (1/5) | 3 μl |
| PCR Ready mix | 10 μl |
| EcoRI primer + A(10 μM) | 0.8 μl |
| HpaII/MspI primer + A | 0.8 μl |
| (10 μM) | |
| Water | 5.4 μl |

TABLE 13
Pre-amplification PCR program

| | | |
|---|---|---|
| 95° C. | 10 mins | |
| 95° C. | 30 sec | 20 cycles |
| 60° C. | 1 min | |
| 72° C. | 1 min | |
| 72° C. | 2 min | |

TABLE 14
Selective amplification primers

| EcoRI | HpaII/MspI |
|---|---|
| 5′ AGACTGCGTACCATTCAC 3′ | 5′ GATGAGTCTCGATCGGACT 3′ |
| (SEQ ID NO: 126) | (SEQ ID NO: 127) |
| 5′ AGACTGCGTACCATTCAA 3′ | 5′ GATGAGTCTCGATCGGAAT 3′ |
| (SEQ ID NO: 128) | (SEQ ID NO: 129) |
| 5′ AGACTGCGTACCATTCAG 3′ | 5′ GATGAGTCTCGATCGGATC 3′ |
| (SEQ ID NO: 130) | (SEQ ID NO: 131) |
| 5′ AGACTGCGTACCATTCAT 3′ | |
| (SEQ ID NO: 132) | |

TABLE 15
Selective amplification PCR mix

| | |
|---|---|
| Pre-amp DNA (1/15) | 5 μl |
| PCR Ready mix | 10 μl |
| EcoRI primer + AX (10 μM) | 0.8 μl |
| HpaII/MspI primer + AXX | 1 μl |
| (1 μM) | |
| Water | 3.2 μl |

TABLE 16
Touchdown PCR program for selective amplification.

| | | |
|---|---|---|
| 95° C. | 10 mins | |
| 95° C. | 30 sec | 12 cycles |
| 60° C. down 0.7° C. per cycle | 30 sec | |
| 72° C. | 1 min | |
| 95° C. | 30 sec | 23 cycles |
| 56.6° C. | 1 min | |
| 72° C. | 1 min | |
| 72° C. | 2 min | |

The products of the selective amplification step were run on Applied Biosystems Genetic Analyzer. The results were visualised and interpreted using GeneMapper analysis software and exported into Microsoft Excel for further analysis.

Each band within the AFLP protocol was considered to be a single allele of a single locus. For each treatment, the allele identity for each locus was first assigned in a simple qualitative manner 1 (present) or 0 (absent) for each replicate individual. A locus was considered to differ between pairs of stress treatments or between the control and a stress treatment if the allelic profile of individuals for the locus differed by three or more individuals (e.g. 11111111 versus 11111000 would be considered to differ whereas 11111111 vs 00111111 would not). Multivariate analysis (Principal Co-ordinate analysis) was carried out using GenAlex (http://www.kovcomp.co.u/mvsp/).

As a result of the dominant nature of AFLP markers, part of the epigenetic variation between individuals is not captured in presence absence scores (for instance, because of cell type-specific methylation changes). However, these changes might contribute to meaningful variation in fragment peak intensities. Although the relationship between initial fragment copy number and peak height is not linear (for instance, because of PCR steps in the AFLP protocol) (Rodriguez Lopez et al 2004, Verhoeven et al 2009), intensity data may contain at least some biological information on epigenetic variation that can be captured using quantitative analysis (Castiglioni et al., 1999; Klahr et al., 2004). A second approach was therefore used to analyze quantitatively a smaller set of MS-AFLP markers (monomorphic in presence/absence scoring) for which fragment intensity scores were obtained using GeneMapper_software. Raw intensity scores were normalized by dividing each fragment peak height score by the total fluorescence value of all fragments obtained from each sample. This normalization accounts for overall differences in intensity scores between samples, for instance as a result of slight differences between samples in initial DNA concentrations. Normalized intensities were subjected to principal component analysis using Minitab 15 (http://www.minitab.com/en-GB/default.apsx?WT.srch=1&WT.mc_id=SE004815).

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

### REFERENCES

1. Lake, J. A., & Woodward, F. I. Response of stomatal numbers to CO₂ and humidity: control by transpiration rate and abscisic acid. New Phytol. 179, 397-404 (2008).
2. Pilliterri, L. J., Sloan, D. B., Bogenschutz, N. L. & Torii, K. U. Termination of asymmetric cell division and differentiation of stomata. Nature 445, 501-505 (2007).
3. Hetherington, A. M. & Woodward, F. I. The role of stomata in sensing and driving environmental change. Nature 424, 901-908 (2003).
4. Miyazawa, S-I., Livingston, N. J. & Turpin, D. H. Stomatal development in new leaves is related to the stomatal conductance of mature leaves in poplar (Populus trichocarpa x P. deltoides). J. of Exp. Bot. 57, 373-380 (2006).
5. Sekiya, N. & Yano, K. Stomatal density of cowpea correlates with carbon isotope discrimination in different phosphorus, water and CO₂ environments. New Phytol. 179, 799-807 (2008).
6. Berger, D. & Altmann, T. A subtilisin-like serine protease involved in the regulation of stomatal density and distribution in Arabidopsis thaliana. Genes Dev. 14, 1119-1131 (2000).
7. von Groll, U., Berger, D. & Altmann, T. The subtilisin-like serine protease SDD1 mediates cell-to-cell signaling during Arabidopsis stomatal development. Plant Cell 14, 1527-1539 (2002).
8. Nadeau, J. A. & Sack, F. D. Control of stomatal distribution on the Arabidopsis leaf surface. Science 296, 1697-1700 (2002).
9. Bergmann, D. C., Lukowitz, W. & Somerville, C. R. Stomatal development and pattern controlled by a MAPKK kinase. Science 304, 1494-1497 (2004).
10. Shpak, E. D., McAbee, J. M., Pillitteri, L. J. & Torii, K. U. Stomatal patterning and differentiation by synergistic interactions of receptor kinases. Science 309, 290-293 (2005).
11. MacAlister, C. A., Ohashi-Ito, K. & Bergmann, D. C. Transcription factor control of asymmetric cell divisions that establish the stomatal lineage. Nature 445, 537-540 (2007).
12. Molinier, J., Ries, G., Zipfel, C. & Hohn, B. Transgeneration memory of stress in plants. Nature 442, 1046-1049 (2006).
13. Jones, L., Ratcliff, F. & Baulcombe, D.C. RNA-directed transcriptional gene silencing in plants can be inherited independently of the RNA trigger and requires Met1 for maintenance. Current Biology. 11, 747-757 (2001).
14. Saze, H., Scheid, O. M. & Paszkowski, J. Maintenance of CpG methylation is essential for epigenetic inheritance during plant gametogenesis. Nature Genetics. 34, 65-69 (2003).
15. Mathieu, 0., Reinders, J., \check{C}aikovski, M., Smathajitt, C. & Paszkowski, J. Transgenerational stability of the Arabidopsis epigenome is coordinated by CG methylation. Cell. 130, 851-862 (2007).
16. Zhang, X. et al. Genome-wide high-resolution mapping and functional analysis of DNA methylation in Arabidopsis. Cell 126, 1189-1201 (2006).
17. Zilberman, D., Gehring, M., Tran, R. K., Ballinger, T. & Henikoff, S. Genome-wide analysis of Arabidopsis thaliana DNA methylation uncovers an interdependence between methylation and transcription. Nature Genet. 39, 61-69 (2007).
18. Cokus, S. J. et al. Shotgun bisulphite sequencing of the Arabidopsis genome reveals DNA methylation patterning. Nature 452, 215-219 (2008).
19. Lister, R. et al. Highly integrated single-base resolution maps of the epigenome in Arabidopsis. Cell. 133, 523-536 (2008).
20. Ohashi-Ito, K. & Bergmann, D. C. Arabidopsis FAMA controls the final proliferation/differentiation switch during stomatal development. Plant Cell 18, 2493-2505 (2006).
21. Finnegan, E. J. & Dennis, E. S. Isolation and identification by sequence homology of a putative cytosine methyltransferase from Arabidopsis thaliana. Nucl. Acids Res. 21, 2383-2388 (1993).
22. Kishimoto, N. et al. Site specificity of the Arabidopsis MET1 DNA methyltransferase demonstrated through hypermethylation of the superman locus. Plant Mol. Biol. 46, 171-183 (2001).
23. Cao, X. & Jacobsen, S. E. Role of the Arabidopsis DRM methyltransferases in de novo DNA methylation and gene silencing. Curr. Biol. 12, 1138-1144 (2002).
24. Cao, X. et al. Role of the DRM and CMT3 methyltransferases in RNA-directed DNA methylation. Curr. Biol. 13, 2212-2217 (2003).
25. Lindroth, A. M. et al. Requirement of CHROMOMETHYLASE3 for maintenance of CpXpG methylation. Science. 292, 2077-2080 (2001).
26. Bartee, L., Malagnac, F. & Bender, J. Arabidopsis cmt3 chromomethylase mutations block non-CG methylation and silencing of an endogenous gene. Genes Dev. 15, 1753-1758 (2001).
27. Wassenegger, M., Heimes, S., Riedel, L. & Sanger, H. L. RNA-directed de novo methylation of genomic sequences in plants. Cell. 76, 567-576 (1994).
28. Wassenegger, M. & Pélissier, T. A model for RNA-mediated gene silencing in higher plants. Plant Mol. Biol. 37, 349-362 (1998).
29. Chan, S. W.-L. et al. RNA silencing genes control de Novo DNA methylation. Science 303, 1336 (2004).
30. Daxinger, L. et al. A stepwise pathway for biogenesis of 24-nt secondary siRNAs and spreading of DNA methylation. EMBO J. 28, 48-57 (2009).
31. Mette, M. F., Aufsatz, W., van der Winden, J., Matzke, M. A. & Matzke, A. J. M. Transcriptional silencing and promoter methylation triggered by double-stranded RNA. EMBO J. 19, 5194-5201 (2000).
32. Vaistij, F. E., Jones, L. & Baulcombe, D. C. Spreading of RNA targeting and DNA methylation in RNA silencing requires transcription of the target gene and a putative RNA-dependent RNA polymerase. Plant Cell. 14, 857-867 (2002).
33. Xie, Z. et al. Genetic and functional diversification of small RNA pathways in plants. PloS Biol. 2, 0642-0652 (2004).
34. Eamens, A., Vaistij, F. E. & Jones, L. NRPD1a and NRPD1b are required to maintain post-transcriptional RNA silencing and RNA-directed DNA methylation in Arabidopsis. Plant Journal. 55, 596-606 (2008).
35. Gasciolli, V., Mallory, A. C., Bartel, D. P. & Vaucheret, H. Partially redundant functions of Arabidopsis DICER-like enzymes and a role for DCL4 in producing trans-acting siRNAs. Curr. Biol. 15, 1494-1500 (2005).
36. Kasschau, K. D. et al. Genome-wide profiling and analysis of Arabidopsis siRNAs. PLoS Biol. 5, e57 (2007).
37. Rajagopalan, R., Vaucheret, H., Trejo, J. & Bartel, D. P. A diverse and evolutionarily fluid set of microRNAs in Arabidopsis thaliana. Genes Dev. 20, 3407-3425 (2006).
38. Axtell, M. J., Jan, C., Rajagopalan, R. & Bartel, D. P. A two-hit trigger for siRNA biogenesis in plants. Cell 127, 565-577 (2006).
39. Fahlgren, N. et al. High-throughput sequencing of Arabidopsis microRNAs: Evidence for frequent birth and death of MIRNA Genes. PLoS ONE 2, e219 (2007).
40. Howell, M. D. et al. Phasing patterns and genome-wide profiles of RDR6/DCL4-dependent small RNAs in Arabidopsis. Plant Cell 19, 926 (2007).
41. Swarbreck, D. et al. The Arabidopsis Information Resource (TAIR): gene structure and function annotation. Nucleic Acids Res. 36, D1009-D1014 (2008).
42. Tran, R. K. et al. Chromatin and siRNA pathways cooperate to maintain DNA methylation of small transposable elements in Arabidopsis. Genome Biol. 6, R90 doi:10.1186/gb-2005-6-11-r90 (2005).
43. Henderson, I. R. & Jacobsen, S. E. Tandem repeats upstream of the Arabidopsis endogene SDC recruit non-CG methylation and initiate siRNA spreading. Genes & Dev. 22, 1597-1606 (2008).
44. Soppe, W. J et al. The late flowering phenotype of fwa mutants is caused by gain-of function epigenetic alleles of a homeodomain gene. Mol. Cell 6, 791-802 (2000).
45. Lippmann, Z. et al. Role of transposable elements in heterochromatin and epigenetic control. Nature 430, 471-476 (2004).
46. Kinoshita, Y. et al. Control of FWA gene silencing in Arabidopsis thaliana bp SINE-related direct repeats. Plant Journal 49, 38-45 (2006).
47. Chan, S. W.-L., Zhang, X., Bernatavichute, Y. V. & Jacobsen, S. E. Two-step recruitment of RNA-directed DNA methylation to tandem repeats. PloS Biol. 4, e363 (2006).
48. Texeira, F. K. et al. A role for RNAi in the selective correction of DNA methylation defects. Science 323, 1600-1604 (2009).
49. Gehring, M., Bubb, K. L. & Henikoff, S. Extensive demethylation of repetitive elements during seed development underlies gene imprinting. Science 324, 1447-1451 (2009).
50. Hsieh, T.-F. et al. Genome-wide demethylation of the Arabidopsis endosperm. Science 324, 1451-1454 (2009).
51. Penterman, J. et al. DNA demethylation in the Arabidopsis genome. PNAS 104, 6752-6757 (2007).
52. Ponferrada-Marin, M. I., Roldán-Arjona, T. & Ariza, R.R. ROS1 5-methylcytosine DNA glycosylase is a slow-turnover catalyst that initiates DNA demethylation in a distributive pattern. Nucleic Acids Res. 37, 4264-4274 (2009).
53. Lunerová-Bed\check{r}ichova, J. et al. Trans-generation inheritance of methylation patterns in a tobacco transgene following a post-transcriptional silencing event. Plant Journal 54, 1049-1062 (2008).
54. Kanaoka, M. M. et al. SCREAM/ICE1 and SCREAM2 specify three cell-state transitional steps leading to Arabidopsis stomatal differentiation. Plant Cell 20, 1775-1785 (2008).
55. Lampard, G. R., Lukowitz, W., Ellis, B. E. & Bergmann, D. C. Novel and expanded roles for MAPK signaling in Arabidopsis stomatal cell fate revealed by cell type-specific manipulations. Plant Cell 21, 3506-3517 (2009).
56. Whittle, C. A., Otto, S. P., Johnston, M. O. & Krochko, J. E. Adaptive epigenetic memory of ancestral temperature regime in Arabidopsis thaliana. Botany 87, 650-657 (2009).
57. Boyes, D. C. et al. Growth stage-based phenotypic analysis of Arabidopsis: a model for high-throughput functional genomics in plants. Plant Cell 13, 1499-1510 (2001).
58. Wojdacz, T. K. & Dobrovic, A. Methylation-sensitive high resolution melting (MS-HRM): a new approach for sensitive and high-throughput assessment of methylation. Nucleic Acids Res. 35, No. 6 e41
59. White, H. E., Hall, V. J. & Cross, N. C. P. Methylation sensitive high-resolution melting-curve analysis of the SNRPN gene as a diagnostic screen for Prader-Willi and Angleman syndromes. Clinical Chemistry 53, 1960-1975 (2007).
60. Ordway, J. M. et al. Identification of novel high-frequency DNA methylation changes in breast cancer. PloS ONE 2, el 314. doi:10.1371/journal.pone.0001314 (2007).
61. Stanley, K.K. & Szewczuk, E. Multiplexed tandem PCR: gene profiling from small amounts of RNA using SYBR green detection. Nucleic Acids Res. 33, 20 e180 (2005).
62. Czechowski, T., Stitt, M., Altmann, T., Udvardi, M. K. & Scheible, W-R. Genome-wide identification and testing of superior reference genes for transcript normalization in Arabidopsis. Plant Physiol. 139, 5-17 (2005).
63. Larkin, M. A. et al. Clustal W and Clustal X version 2.0. Bioinformatics 23, 2947-2948 (2007).
64. Hall, T. A. BioEdit: a user-friendly biological sequence alignment editor and analysis program for Windows 95/98/NT. Nucl. Acids. Symp. Ser. 41, 95-98 (1999).
65. Li, L. C. & Dahiya, R. MethPrimer: designing primers for methylation PCRs. Bioinformatics 18, 1427-31 (2002).

1. A method for the production of a stress tolerant plant or precursor thereof, the method comprising:
(i) subjecting one or more parental plants to one or more stress conditions selected from unfavourable conditions relating to relative humidity, water availability, periodic drought, nutrients, sunlight, wind, temperature, pH, exogenous chemicals, chemical toxins including salt, herbivory, prophylactic chemicals, fertilizers, pathogen attack including bacterial, fungal, or virus infection and pest infestation; and
(ii) generating offspring from said one or more parental plants,
wherein said offspring show enhanced tolerance relative to the one or more parental plants to one or more stress conditions selected from unfavourable conditions relating to relative humidity, water availability, periodic drought, nutrients, sunlight, wind, temperature, pH, exogenous chemicals, chemical toxins including salt, herbivory, prophylactic chemicals, fertilizers, pathogen attack including bacterial, fungal, or virus infection and pest infestation.

2. The method according to claim 1, wherein the offspring are adult plants or a precursor thereof.

3. The method according to claim 1, wherein the offspring are seeds or vegetative propagules.

4. The method according to claim 1, wherein the offspring are tolerant to one or more stress conditions experienced by the one or more parental plants.

5. The method according to claim 1, wherein the offspring are tolerant to one or more stress conditions not experienced by the one or more parental plants.

6. The method according to claim 1, wherein one or more parental plants are subjected to one or more stress conditions selected from low relative humidity, periodic drought and infection with Botrytis and/or wherein said offspring show enhanced tolerance to one or more stress conditions selected from low relative humidity, periodic drought and infection with Botrytis.

7. The method according to claim 1, wherein the one or more parental plants are selected from higher plants, flowering plants and dicotyledonous plants.

8. The method according to claim 1, wherein the one or more parental plants are crop plants.

9. The method according to claim 1, wherein the one or more parental plants belong to the Eudicotyledons, including the Brassicacea or the Malvaceae family.

10. The method according to claim 1, wherein the offspring show increased production of biomass, flower number, seed number and/or seed weight at any chosen time of harvest relative to the one or more parental plants.

11. A plant, or precursor thereof, produced by a method according to claim 1.

12. An assay for identifying a plant, or precursor thereof, produced by a method according to claim 1, wherein the assay comprises analysing a plant, or precursor thereof, suspected of being produced by the method for the presence or absence of one or more sites of genomic methylation, wherein the presence or absence of methylation at said one or more sites is indicative of a plant, or precursor thereof, produced by the method.

13. The assay according to claim 12, wherein the presence of genomic methylation at or within about 10 kb of a SPEECHLESS or FAMA gene, or a functional homolog of either gene, is indicative of a plant, or precursor thereof, which is tolerant to low relative humidity and/or periodic drought.

14. An assay for identifying a plant, or precursor thereof, which is tolerant to one or more stress conditions selected from unfavourable conditions relating to relative humidity, water availability, periodic drought, nutrients, sunlight, wind, temperature, pH, exogenous chemicals, chemical toxins including salt, herbivory, prophylactic chemicals, fertilizers, pathogen attack including bacterial, fungal, or virus infection and pest infestation, wherein the assay comprises analysing a plant, or precursor thereof for the presence or absence of one or more sites of genomic methylation, wherein the presence or absence of methylation at said one or more sites is indicative of a plant, or precursor thereof, which is tolerant to said one or more stress conditions.

15. The assay according to claim 14, wherein the presence of genomic methylation at or within 10 kb of a SPEECHLESS or FAMA gene, or a functional homolog of either gene, is indicative of a plant, or precursor thereof, which is tolerant to low relative humidity and/or periodic drought.
